SEQUENCE LISTING (11 sequences)

SEQ ID NO: 1 - protein, 139 aa, Artificial Sequence
  Description of Artificial Sequence Synthetic polypeptide
  MOD_RES (1)..(1): V, G, L, A, I, T, Y, or F
  MOD_RES (2)..(2): L, G, A, I, M, Y, or F
  MOD_RES (3)..(3): Q, D, N, W, H, M, S, R, or K
  MOD_RES (22)..(22): R, H, D, E, N, M, or Q
  MOD_RES (26)..(26): S, T, or A
  MOD_RES (38)..(38): R, T, H, D, E, N, M, or Q
  MOD_RES (69)..(69): L, G, A, I, M, Y, or F
  MOD_RES (82)..(82): G, R, A, V, L, or I
  MOD_RES (84)..(84): K or absent
  MOD_RES (110)..(110): H, D, E, N, M, or Q
XXXKFVGTWKIADSHNFGEYLXAIGXPKELSDGGDATXPTLYISQKDGDKMTVKIENGPPTFLDTQVKXKLGEEFDEFPSDXRXGVKSVVNLVGEKLVYVQKWDGKETTXVREIKDGKLVVTLTMGDVVAVRSYRRATE

SEQ ID NO: 2 - protein, 139 aa, Artificial Sequence
  Description of Artificial Sequence Synthetic polypeptide
VLQKFVGTWKIADSHNFGEYLRAIGSPKELSDGGDATRPTLYISQKDGDKMTVKIENGPPTFLDTQVKLKLGEEFDEFPSDGRKGVKSVVNLVGEKLVYVQKWDGKETTHVREIKDGKLVVTLTMGDVVAVRSYRRATE

SEQ ID NO: 3 - protein, 139 aa, Artificial Sequence
  Description of Artificial Sequence Synthetic polypeptide
VLQKFVGTWKIADSHNFGEYLRAIGAPKELSDGGDATTPTLYISQKDGDKMTVKIENGPPTFLDTQVKFKLGEEFDEFPSDRRKGVKSVVNLVGEKLVYVQKWDGKETTHVREIKDGKLVVTLTMGDVVAVRSYRRATE

SEQ ID NO: 4 - protein, 83 aa, Artificial Sequence
  Description of Artificial Sequence Synthetic polypeptide
  MOD_RES (1)..(1): V, G, L, A, I, T, Y, or F
  MOD_RES (2)..(2): L, G, A, I, M, Y, or F
  MOD_RES (3)..(3): Q, D, N, W, H, M, S, R, or K
  MOD_RES (22)..(22): R, H, D, E, N, M, or Q
  MOD_RES (26)..(26): S, T, or A
  MOD_RES (38)..(38): R, T, H, D, E, N, M, or Q
  MOD_RES (69)..(69): L, G, A, I, M, Y, or F
  MOD_RES (82)..(82): G, R, A, V, L, or I
XXXKFVGTWKIADSHNFGEYLXAIGXPKELSDGGDATXPTLYISQKDGDKMTVKIENGPPTFLDTQVKXKLGEEFDEFPSDXR

SEQ ID NO: 5 - protein, 83 aa, Artificial Sequence
  Description of Artificial Sequence Synthetic polypeptide
VLQKFVGTWKIADSHNFGEYLRAIGSPKELSDGGDATRPTLYISQKDGDKMTVKIENGPPTFLDTQVKLKLGEEFDEFPSDGR

SEQ ID NO: 6 - protein, 83 aa, Artificial Sequence
  Description of Artificial Sequence Synthetic polypeptide
VLQKFVGTWKIADSHNFGEYLRAIGAPKELSDGGDATTPTLYISQKDGDKMTVKIENGPPTFLDTQVKFKLGEEFDEFPSDRR

SEQ ID NO: 7 - protein, 55 aa, Artificial Sequence
  Description of Artificial Sequence Synthetic polypeptide
  MOD_RES (26)..(26): H, D, E, N, M, or Q
GVKSVVNLVGEKLVYVQKWDGKETTXVREIKDGKLVVTLTMGDVVAVRSYRRATE

SEQ ID NO: 8 - protein, 55 aa, Artificial Sequence
  Description of Artificial Sequence Synthetic polypeptide
GVKSVVNLVGEKLVYVQKWDGKETTHVREIKDGKLVVTLTMGDVVAVRSYRRATE

SEQ ID NO: 9 - protein, 4 aa, Artificial Sequence
  Description of Artificial Sequence Synthetic linker peptide
GGSA

SEQ ID NO: 10 - DNA, 249 bp, Artificial Sequence
  Description of Artificial Sequence Synthetic polynucleotide
gtgctccagaaattcgtaggaacttggaagatagcagattcacataatttcggcgaatatctcagagccataggagcaccaaaggaattatcagatggcggtgatgccacaactcccactctgtatatcagccagaaagatggcgacaaaatgacggtgaaaatagagaacggcccaccgaccttcctggacactcaggtgaagtttaaactgggtgaggagtttgacgagtttccttctgacgggcgt

SEQ ID NO: 11 - DNA, 165 bp, Artificial Sequence
  Description of Artificial Sequence Synthetic polynucleotide
ggtgttaaaagcgtcgttaacctggtgggagagaaattagtatacgtccaaaagtgggacggcaaggagactacgcacgttagagagattaaagacggcaagctggtggtaacactaacaatgggcgacgtcgttgcagtgcgctcatatcgcagggcgacggag

(19) United States
(12) Patent Application Publication
(10) Pub. No.: US 2024/0248079 A1
(43) Pub. Date: Jul. 25, 2024

(54) IMPROVED UnaG FLUORESCENT PROTEIN FOR BiFC ASSAYS

(71) Applicant: THE REGENTS OF THE UNIVERSITY OF CALIFORNIA, Oakland, CA (US)

(72) Inventors: Xiaokun Shu, San Francisco, CA (US); Junjiao Yang, San Francisco, CA (US)

(21) Appl. No.: 18/562,161

(22) PCT Filed: May 16, 2022
(86) PCT No.: PCT/US22/29492

§ 371 Date: Nov. 17, 2023

Related U.S. Application Data
(60) Provisional application No. 63/189,370, filed on May 17, 2021.

(51) Int. Cl. G01N 33/50; G01N 33/58

(52) U.S. Cl. CPC G01N 33/5091; G01N 33/5017; G01N 33/582

(57) ABSTRACT

By directed evolution, various amino acid substitutions which impart greater brightness to the UnaG fluorescent protein were developed. With certain combinations of mutations, the improved UnaG protein has brightness 100 times greater than the original parent sequence. Bi-molecular fluorescence complementation assays using the improved UnaG variants provide strong signal and high resolution and provide powerful tools for detecting protein-protein interactions (PPIs). PPI detection tools for various important proteins are provided. These assays enable highly efficient screening of putative PPI modulators and the identification, verification, and development of therapeutics that disrupt pathological PPIs.

### CROSS-REFERENCE TO RELATED APPLICATIONS

This application claims the benefit of priority to U.S. Provisional Patent Application Ser. No. 63/189,370 entitled “Improved UnaG Fluorescent Protein for BiFC Assays,” filed May 17, 2021, the contents of which are hereby incorporated by reference.

### REFERENCE TO SEQUENCE LISTING

A sequence listing is submitted herewith as “Sequence Listing,” pages 38-41.

### STATEMENT REGARDING FEDERALLY SPONSORED RESEARCH OR DEVELOPMENT

Not applicable.

### BACKGROUND OF THE INVENTION

Interactions between proteins underlie just about all biological process. Protein-protein interactions (PPIs) are defined as the physical contact between two or more proteins. Examples of PPIs include interactions between receptors and ligands, host-pathogen interactions, cell signaling pathways and cascades, and enzymatic reactions essential to metabolism. Accordingly, measurement of PPIs is of critical importance in the field of biological and medical research.

One of the more powerful tools developed for the detection of PPIs is the bimolecular fluorescence complementation (BiFC) assay. Typical BiFC assays utilize a split fluorescent protein, wherein the protein is split into two fragments, and wherein by such division the chromophore structure is disrupted such that each of the two fragments is not itself fluorescent, or is substantially less fluorescent than the intact protein. Each of the two fragments is fused to a protein that participates, or putatively participates, in a PPI (the two proteins being referred to herein as “PPI partners”). If the two fluorescent protein fragments are brought into proximity by a PPI between the two PPI partners they are fused to, the chromophore is reconstituted, creating fluorescent signal in response to illumination by suitable wavelengths of light.

BiFC assays have been developed using a large number of fluorescent proteins, such as green fluorescent protein, yellow fluorescent protein, red fluorescent proteins, and others. In recent years, a novel fluorescent protein, UnaG, was isolated from the Japanese freshwater eel (Anguilla japonica) as described in Kumagai et al. 2013. A bilirubin-inducible fluorescent protein from eel muscle. Cell 153:1602-1611. This unique fluorescent protein incorporates endogenous bilirubin (BR) as the chromophore. Bilirubin is a ubiquitous yellowish pigment that is produced as a byproduct of red blood cell breakdown, and is not itself fluorescent. When illuminated with blue light, e.g. at wavelengths of 488 nm, UnaG fluoresces green with peak emissions at about 527 nm.

In a previous report by one of the inventors of the present disclosure, a BiFC assay utilizing UnaG was reported, as described in To et al., 2016. Structure-guided design of a reversible fluorogenic reporter of protein-protein interactions, Protein Sci. 25: 748-753. In that previous work, the crystal structure of UnaG was utilized to guide the design of the BiFC assay. A region between amino acid residues 72 and 85 forming a loop atop the chromophore was identified. Splits were made at 72 (N-terminal end of the loop), 78 (middle of the loop), and 84 (C-terminal end of the loop). Best results were obtained with the split at position 84. The resulting BiFC assay had good signal-to-noise ratio, rapid onset kinetics, and, advantageously, was reversible, enabling detection of reversible PPIs. PPI detection was achieved without the use of exogenous factors.

Another effort to improve UnaG is described in Zharadnik et al., 2020, An enhanced yeast display platform demonstrates the binding plasticity under various selection pressures, bioRxiv preprint 10.1101/2020.12.16.423176, wherein mutations that impart greater stability and signal to the UnaG sequence are disclosed.

Despite the value of these previous efforts to improve the UnaG BiFC assay, there remains a need in the art for improved BiFC assays with improved signal-to-noise, more rapid onset, and improved versatility across a broad range of biological systems.

### SUMMARY OF THE INVENTION

The inventors of the present disclosure have advantageously developed improved BiFC assays. By directed mutagenesis of the UnaG protein, the inventors of the present disclosure discovered multiple amino acid substitutions in UnaG that dramatically improve the fluorescence of the protein. The novel mutants, in certain combinations, improve the brightness of the UnaG assay by over a factor of 100. A novel PPI assay system based upon this improved fluorescent protein is termed “SURF” for Split UnaG-based Reversible and Fluorogenic PPI reporter). As described herein, SURF advantageously enables detection of PPIs from diverse protein types, including for example, interaction between a G protein-coupled receptor (GPCR) and beta arrestin upon addition of GPCR agonist, between E3 ubiquitin ligase and its substrate, between a small GTPase and its effector, between transcription factors, and between a transcription factor and its interacting kinase. When compared to other PPI reporter systems, SURF was found to have large dynamic range, high brightness, fast on and off kinetics, in addition to advantageously being genetically encoded and requiring no exogenous cofactors. Accordingly, SURF provides the art with a novel and versatile BiFC with numerous advantages over the prior art.

In a first aspect, the scope of the invention encompasses novel compositions of matter comprising engineered variants of UnaG having improved brightness. In one aspect, the scope of the invention encompasses complementary fragments of the foregoing UnaG variants, for use in a BiFC assay.

In another aspect, the scope of the invention encompasses a novel PPI assay system based upon the foregoing improved fluorescent protein. In a related aspect, the scope of the invention encompasses methods of using the novel PPI assays described herein to detect, measure, and/or quantify a selected PPI. In a related aspect, the scope of the invention encompasses a screening method for identifying modulators of a PPI detected by the improved systems of the invention.

The various elements of the invention are described next.

### BRIEF DESCRIPTION OF THE DRAWINGS

FIG. 1. FIG. 1 is a diagram of the SURF system based on engineered UnaG proteins of the invention. To visualize the interaction between two proteins X and Y, a first fragment of the engineered UnaG protein of the invention (cSURF: carboxyl-terminal fragment) is fused to protein X; and a second, complementary fragment of the engineered UnaG protein (nSURF: amino-terminal fragment) is fused to protein Y. When the two proteins interact, this brings the two fragments into close proximity so that the engineered UnaG fluorescent protein reconstitutes and becomes fluorescent. PPI inhibition dissociates two interacting proteins, resulting in dissociation of the two SURF fragments and loss of fluorescence.

FIG. 2. FIG. 2. depicts an exemplary SURF system for quantifying the PPI between proteinase-activated receptor 1 (Par1) and β-arrestin. A BiFC assay comprising cSURF (SEQ ID NO: 8) fused to Par1 and nSURF (SEQ ID NO: 6) fused to β-arrestin was expressed in a cell. Upon addition of a Par1 agonist, the PPI was induced. Fluorescent microscopy scans across the width of the cell at 0, 3, and 6.5 minutes were performed. Localized fluorescence was observed at the cell periphery, indicative of the Par1—β-arrestin PPI occurring in the membrane. Meanwhile, co-expressed RFP was stable across the cell.

FIGS. 3A and 3B. FIGS. 3A and 3B depict on- and off-kinetics of two implementations of the SURF assay. FIG. 3A depicts results using a SURF assay comprising cSURF (SEQ ID NO: 8) fused to Par1 and nSURF (SEQ ID NO: 6) fused to β-arrestin. A Par1 agonist was administered to the cell at time 0, resulting in rapid onset of fluorescence with a T_{1/2} ON time of about 3 minutes. FIG. 3B depicts results using a SURF assay comprising cSURF (SEQ ID NO: 6) fused to the transactivation domain of p53 (p53^{TAD}) and nSURF (SEQ ID NO: 4) fused to the p53 binding domain of oncoprotein MDM2 (MDM253^{p53BD}). Nutlin-3a, which induces dissociation between the PPI partners, was administered to the cell at time 0, resulting in rapid decline of UnaG fluorescence with a T_{1/2} OFF time of about five minutes.

FIGS. 4A, 4B, and 4C. FIGS. 4A, 4B, and 4C depict the results of high throughput screens to identify modulators of various PPIs. FIG. 4A depicts results using a SURF system engineered to measure the interaction between KRas^{G12V} and Raf1, wherein cSURF was fused to the receptor binding domain of Raf1 (Raf1RBD) and nSURF was fused to KRas^{G12V}. SURF visualized this interaction with green fluorescence on the plasma membrane. A high throughput screen of 1622 FDA-approved drugs (1 μM final concentration) was performed. Six clinical drugs were identified, showing 50%-80% inhibition, left side of volcano plot, as well as three drugs that increase the interaction, right side of the plot. FIG. 4B depicts results using a SURF system engineered to measure the interaction between YAP1 and TEAD, wherein cSURF was fused to YAP1 and nSURF was fused to TEAD4. A high throughput screen of 1622 FDA-approved drugs (1 μM final concentration) was performed. Eight clinical drugs were identified that showed 50%-70% inhibition, left side of volcano plot, and three drugs were identified that increase the interaction, right side of the plot. FIG. 4C. depicts results using a SURF system engineered to measure the interaction between MYCN and AURKA, wherein cSURF was fused to the 137 n-terminal amino acids of MYCN and nSURF was fused to the kinase domain of Aurora Kinase A. A high throughput screen of 1622 FDA-approved drugs (1 μM final concentration) was performed. Six clinical drugs significantly inhibited this PPI, shown in the left side of the volcano plot.

FIG. 5. FIG. 5 depicts SURF sensitivity to modulators of PPIs. In this experiment, MYCN was fused to cSURF (SEQ ID NO: 8) and Aurora kinase A was fused to nSURF (SEQ ID NO: 6). In DMSO controls, the PPI produced stable fluorescent signal across a 24 hour measurement window. Upon addition of the PPI inhibitor CD532, substantially inhibited SURF fluorescence over time. Addition of MLN8237, a mild disruptor of the PPI, resulted in about 5% attenuation of SURF fluorescence. Addition of VX680, which does not disrupt the PPI did not alter fluorescent signal of the PPI reporter.

FIG. 6. FIG. 6 depicts follow-up verification of MYCN-AURKA inhibitors identified in the high throughput screen depicted in FIG. 4C.

FIGS. 7A and 7B. FIGS. 7A and 7B depict the inhibition kinetics of the MYCN-AURKA inhibitors measured using cSURF and nSURF identified in the high throughput screen depicted in FIG. 4C.

FIG. 8. FIG. 8 depicts MYCN protein levels in cancer cell lines, measured by western blot, following administration of inhibitors MYCN-AURKA inhibitors identified in the high throughput screen depicted in FIG. 4C.

FIG. 9. FIG. 9 depicts a SURF PPI reporter comprising FKBP fused to the cSURF fragment (SEQ ID NO: 8) and Frb protein fused to nSURF (SEQ ID NO: 6). Upon addition of rapyamycin, a PPI is initiated and strong signal was generated by the fragments of the engineered UnaG protein. A comparable assay using the previously reported UPPI construct was run in parallel, and the SURF assay of the invention had brightness that was at least 100 times greater.

FIG. 10. FIG. 10 depicts signal brightness of the cpUnaG protein through five rounds of directed evolution, wherein brightest colonies were selected in each round.

FIG. 11. FIG. 11 depicts the excitation and emission spectra of the engineered UnaG protein of the invention (SEQ ID NO: 3), with peak excitation at 498 nm and peak emission at 527 nm.

### DETAILED DESCRIPTION OF THE INVENTION

### A. Engineered UnaG.

The novel PPI detection assays disclosed herein utilize an engineered variant of the UnaG protein. The UnaG protein, as known in the art, is described, for example, in Kumagai et al. 2013. A bilirubin-inducible fluorescent protein from eel muscle. Cell 153:1602-1611. The protein is designated NCBI identifier 7937 and Uniprot identifier PODM59. UnaG utilizes the molecule bilirubin as the chromophore. Thus, in cells wherein bilirubin is present, UnaG fluorescence may be achieved without the requirement of an exogenous cofactor. Bilirubin is a tetrapyrrole bilin and free bilirubin is not fluorescent. The various inventions described herein are based on certain improvements of the original UnaG sequence, comprising amino acid substitutions at select residues which impart much greater brightness than original UnaG. It will be understood that reference will be made herein to proteins, polypeptides, and peptides. It will be understood that such species may comprise an amino acid sequence of any length. Reference to a sequence encompasses the sequence itself, and variants thereof, for example, having at least 90%, 95%, or 99% sequence identity thereto.

In one implementation, the scope of the invention encompasses an engineered UnaG protein comprising a protein having at least 90%, at least 95%, or at least 99% sequence identity to SEQ ID NO: 1:

X₁X₂X₃KFVGTWKIADSHNFGEYLX₄AIGX₅PKELSDGGDATX₆P
TLYISQKDGDKMTVKIENGPPTFLDTQVKX₇KLGEEFDEFPSDX₈
RX₉GVKSVVNLVGEKLVYVQKWDGKETTX₁₀VREIKDGKLVVTLT
MGDVVAVRSYRRATE

wherein X1 is V, G, L, A, I, T, Y, or F;
wherein X2 is L, G, A, IM, Y, or F;
wherein X3 is Q, D, N, W, H, M, S, R, or K;
wherein X4 is R, H, D, E, N, M, or Q;
wherein X5 is S, T, or A;
wherein X6 is R, T, H, D, E, N, M, or Q;
wherein X7 is L, G, A, IM, Y, or F;
wherein X8 is G, R, A, V, L, or I
wherein X9 is K or omitted; and
wherein X10 is H, D, E, N, M, or Q.

Reference to amino acids made herein may encompass use of the amino acid's full name, or by its one letter code, as known in the art, for example: Alanine (A), Arginine (R); Asparagine (N); Aspartic acid (D); Cysteine (C); Glutamic acid (E); Glutamine (Q); Glycine (G); Histidine (H); Isoleucine (I), Leucine (L), Lysine (K); Methionine (M); Phenylalanine (F); Proline (P), Serine (S); Threonine (T); Tryptophan (W); Tyrosine (Y); Valine (V).

X1 comprises a substitution of the original methionine at UnaG sequence position 1, for example, an amino acid substitution comprising valine (MIV). X2 comprises substitution of the original valine at UnaG sequence position 2, for example, an amino acid substitution comprising leucine (V2L). X3 comprises substitution of the original glutamic acid at UnaG sequence position 3, for example, an amino acid substitution comprising glutamine (E3Q). X4 comprises substitution of the original lysine at UnaG sequence position 22, for example, an amino acid substitution comprising arginine (K22R). X5 comprises a substitution of the original alanine at UnaG position 26, for example, an amino acid substitution comprising serine. X6 comprises substitution of the original threonine at UnaG sequence position 38, for example, an amino acid substitution comprising arginine (T38R). X7 comprises a substitution of the original phenylalanine at UnaG position 69, for example, an amino acid substitution comprising leucine (F69L). X8 comprises a substitution of the original arginine at UnaG sequence position 82, for example, an amino acid substitution comprising glycine (R82G). X9 comprises lysine as in the original UnaG sequence, or the deletion thereof. X10 comprises substitution of the original tyrosine at UnaG sequence position 110, for example, an amino acid substitution comprising histidine (Y110H). In various embodiments, one or more of the amino acid substitutions X1, X2, X3, X4, X5, X6, X7, X8, X9, or X10 are omitted, wherein, in such implementations, the engineered UnaG comprises a protein comprising at least 90%, at least 95%, or at least 99% sequence identity to SEQ ID NO: 1, wherein any of: X1 may be methionine; X2 may be valine; X3 may be glutamic acid; X4 may be lysine; X5 may be alanine; X6 may be threonine, X7 may be phenylalanine; X8 may be arginine; X9 may be lysine, and X10 may be tyrosine.

In one implementation, the fluorescent protein of the invention comprises a protein wherein all the foregoing substitutions MIV, V2L, E3Q, K22R, A26S, T38R, F69L, R82G, or Y110H to the UnaG protein are utilized. In alternative embodiments, the engineered UnaG protein of the invention comprises only a subset of amino acid substitutions MIV, V2L, E3Q, K22R, A26S, T38R, F69L, R82G, or Y110H, for example, comprising any one of, any two of, any three of, any four of, any five of, any six of, any seven of, or any eight of the amino acid substitutions selected from the group consisting of MIV, V2L, E3Q, K22R, A26S, T38R, F69L, R82G, or Y110H.

In one embodiment, the engineered UnaG protein of the invention comprises a protein and having at least 90%, at least 95%, or at least 99% sequence identity to SEQ ID NO: 2:

VLQKFVGTWKIADSHNFGEYLRAIGSPKELSDGGDATRPTLYISQ
KDGDKMTVKIENGPPTFLDTQVKLKLGEEFDEFPSDGRKGVKSVV
NLVGEKLVYVQKWDGKETTHVREIKDGKLVVTLTMGDVVAVRSYR
RATE.

For example, a protein and having at least 90%, at least 95%, or at least 99% sequence identity to SEQ ID NO: 2 and having the amino acid substitutions MIV, V2L, E3Q, K22R, A26S, T38R, F69L, R82G, or Y110H.

In one embodiment, the engineered Una G protein of the invention comprises an UnaG sequence comprising having at least 90%, at least 95%, or at least 99% sequence identity to SEQ ID NO: 3:

VLQKFVGTWKIADSHNFGEYLRAIGAPKELSDGGDATTPTLYISQ
KDGDKMTVKIENGPPTFLDTQVKFKLGEEFDEFPSDRRKGVKSVV
NLVGEKLVYVQKWDGKETTHVREIKDGKLVVTLTMGDVVAVRSYR
RATE.

For example, a protein and having at least 90%, at least 95%, or at least 99% sequence identity to SEQ ID NO: 3 and having the amino acid substitutions MIV, V2L, E3Q, K22R, and Y110H.

### B. Complementary Fragments of the Engineered UnaG.

The BiFC assay of the invention encompasses complementary fragments of an engineered UnaG sequence of the invention. “Complementary fragments,” as used herein, means fragments (subsequences) of an engineered UnaG sequence, wherein, such fragments alone do not generate significant fluorescent signal when illuminated with suitable wavelengths for the excitation of UnaG, but when such fragments are in sufficient proximity to each other, the fragments will substantially reconstitute the chromophore and the fluorescent properties of the engineered UnaG protein, for example, generating at least 70%, at least 80%, at least 90%, 100%, or greater than 100% of the fluorescent signal as the intact engineered protein, for example, SEQ ID NO: 3.

In a primary embodiment, the BiFC assays of the invention utilize two complementary fragments of the engineered UnaG protein of the invention. In such implementation, the fragments will comprise an n-terminal fragment (denoted “nSURF” herein) and a c-terminal fragment (denoted “cSURF” herein). The fragments may be defined by a “split” site in the complete UnaG sequence. It will be understood that the first and second fragments may, in some embodiments, reconstitute the entire sequence of the engineered UnaG protein, i.e. wherein the split site is between two consecutive amino acids of the engineered protein. In other embodiments, the split encompasses one or more amino acid residues of the protein, which are omitted in the first and second fragments, such that the first and second fragments do not reconstitute the entire engineered UnaG protein.

The split site may be selected at any position of the UnaG protein wherein the resulting fragments, when in sufficient proximity, will reconstitute the fluorescent properties of the UnaG protein. In various embodiments, the split is selected at a position between amino acid residue 70 and amino acid 90 of the UnaG sequence. This section of the protein spans a loop that forms a “lid” over the buried chromophore and provides a region for efficient splitting of the protein. In various embodiments, the split is selected at positions 70/71, 71/72, 72/73, 73/74, 74/75, 75/76, 76/77, 77/78, 78/79, 79/80, 80/81, 81/82, 82/83, 83/84, 84/85, 85/86, 86/87, 87/88, 88/89, 89/90, or 90/91, wherein the slash denotes a split between the enumerated amino acid positions. In some embodiments the split encompasses one or more amino acids, i.e., the amino acid(s) making up the split are omitted from the resulting fragments. In some embodiments the split encompasses a single amino acid, for example, amino acid 70, 71, 72, 73, 74, 75, 76, 77, 78, 78, 79, 80, 81, 82, 83, 84, 85, 86, 87, 88, 89, or 90. In one embodiment, the split site comprises amino acid 84, which is deleted, such that the resulting fragments comprise amino acids 1-83 and 85-139. In some embodiments, the split encompasses two amino acids, for example, 69-70, 70-71, 71-72, 72-73, 73-74, 74-75, 75-76, 76-77, 77-78, 78-79, 79-80, 80-81, 81-82, 82-83, 83-84, 84-85, 85-86, 86-87, 87-88, 88-89, or 89-90. In other implementations, the split encompasses three, four, or more amino acids.

Accordingly, in various embodiments, the nSURF fragment comprises a polypeptide having at least 90%, at least 95% or at least 99% sequence identity to: amino acids 1-70 of SEQ ID NO: 1, SEQ ID NO: 2, or SEQ ID NO: 3; amino acids 1-71 of SEQ ID NO: 1 or SEQ ID NO: 1, SEQ ID NO: 2, or SEQ ID NO: 3; amino acids 1-72 of SEQ ID NO: 1, SEQ ID NO: 2, or SEQ ID NO: 3; amino acids 1-73 of SEQ ID NO: 1, SEQ ID NO: 2, or SEQ ID NO: 3; amino acids 1-74 of SEQ ID NO: 1, SEQ ID NO: 2, or SEQ ID NO: 3; amino acids 1-75 of SEQ ID NO: 1, SEQ ID NO: 2, or SEQ ID NO: 3; amino acids 1-76 of SEQ ID NO: 1, SEQ ID NO: 2, or SEQ ID NO: 3; amino acids 1-77 of SEQ ID NO: 1, SEQ ID NO: 2, or SEQ ID NO: 3; amino acids 1-78 of SEQ ID NO: 1, SEQ ID NO: 2, or SEQ ID NO: 3; amino acids 1-79 of SEQ ID NO: 1, SEQ ID NO: 2, or SEQ ID NO: 3; amino acids 1-80 of SEQ ID NO: 1, SEQ ID NO: 2, or SEQ ID NO: 3; amino acids 1-81 of SEQ ID NO: 1, SEQ ID NO: 2, or SEQ ID NO: 3; amino acids 1-82 of SEQ ID NO: 1, SEQ ID NO: 2, or SEQ ID NO: 3; amino acids 1-83 of SEQ ID NO: 1, SEQ ID NO: 2, or SEQ ID NO: 3; amino acids 1-84 of SEQ ID NO: 1, SEQ ID NO: 2, or SEQ ID NO: 3; amino acids 1-85 of SEQ ID NO: 1 or SEQ ID NO: 2; amino acids 1-86 of SEQ ID NO: 1, SEQ ID NO: 2, or SEQ ID NO: 3; amino acids 1-87 of SEQ ID NO: 1, SEQ ID NO: 2, or SEQ ID NO: 3; amino acids 1-88 of SEQ ID NO: 1, SEQ ID NO: 2, or SEQ ID NO: 3; amino acids 1-89 of SEQ ID NO: 1, SEQ ID NO: 2, or SEQ ID NO: 3; or amino acids 1-90 of SEQ ID NO: 1, SEQ ID NO: 2, or SEQ ID NO: 3. In some implementations of the foregoing embodiments, amino acid 84 is omitted.

Likewise, in various embodiments, the cSURF fragment comprises a polypeptide having at least 90%, at least 95% or at least 99% sequence identity to: amino acids 70-139 of SEQ ID NO: 1, SEQ ID NO: 2, or SEQ ID NO: 3; amino acids SEQ ID NO: 1, SEQ ID NO: 2, or SEQ ID NO: 3: 2; amino acids 72-139 of SEQ ID NO: 1, SEQ ID NO: 2, or SEQ ID NO: 3; amino acids 73-139 of SEQ ID NO: 1, SEQ ID NO: 2, or SEQ ID NO: 3; amino acids 74-139 of SEQ ID NO: 1, SEQ ID NO: 2, or SEQ ID NO: 3; amino acids 75-139 of SEQ ID NO: 1, SEQ ID NO: 2, or SEQ ID NO: 3; amino acids 76-139 of SEQ ID NO: 1, SEQ ID NO: 2, or SEQ ID NO: 3; amino acids 77-139 of SEQ ID NO: 1, SEQ ID NO: 2, or SEQ ID NO: 3; amino acids 78-139 of SEQ ID NO: 1, SEQ ID NO: 2, or SEQ ID NO: 3; amino acids 79-139 of SEQ ID NO: 1, SEQ ID NO: 2, or SEQ ID NO: 3; amino acids 80-139 of SEQ ID NO: 1, SEQ ID NO: 2, or SEQ ID NO: 3; amino acids 81-139 of SEQ ID NO: 1, SEQ ID NO: 2, or SEQ ID NO: 3; amino acids 82-139 SEQ ID NO: 1, SEQ ID NO: 2, or SEQ ID NO: 3; amino acids 83-139 of SEQ ID NO: 1, SEQ ID NO: 2, or SEQ ID NO: 3; amino acids 84-139 of SEQ ID NO: 1, SEQ ID NO: 2, or SEQ ID NO: 3; amino acids 85-139 of SEQ ID NO: 1, SEQ ID NO: 2, or SEQ ID NO: 3; amino acids 86-139 of SEQ ID NO: 1, SEQ ID NO: 2, or SEQ ID NO: 3; amino acids 87-139 of SEQ ID NO: 1, SEQ ID NO: 2, or SEQ ID NO: 3; amino acids 88-139 of SEQ ID NO: 1, SEQ ID NO: 2, or SEQ ID NO: 3; amino acids 89-139 of SEQ ID NO: 1, SEQ ID NO: 2, or SEQ ID NO: 3; or amino acids 90-139 of SEQ ID NO: 1, SEQ ID NO: 2, or SEQ ID NO: 3. In some implementations of the foregoing embodiments, amino acid 84 is omitted.

In a primary embodiment, the nSURF fragment comprises a polypeptide having at least 90%, at least 95% or at least 99% sequence identity to amino acids 1-83 of SEQ ID NO: 1, SEQ ID NO: 2, or SEQ ID NO: 3. In one embodiment, the nSURF fragment comprises a polypeptide having at least 90%, at least 95% or at least 99% sequence identity to amino acids 1-84 of SEQ ID NO: 1, SEQ ID NO: 2 or SEQ ID NO: 3. In one embodiment, the nSURF fragment comprises a polypeptide having at least 90%, at least 95% or at least 99% sequence identity to SEQ ID NO: 4:

X₁X₂X₃KFVGTWKIADSHNFGEYLX₄AIGX₅PKELSDGGDATX₆PTL
YISQKDGDKMTVKIENGPPTFLDTQVKX₇KLGEEFDEFPSDX₈R,

wherein X1 is V, G, L, A, I, T, Y, or F;
wherein X2 is L, G, A, IM, Y, or F;
wherein X3 is Q, D, N, W, H, M, S, R, or K;
wherein X4 is R, H, D, E, N, M, or Q;
wherein X5 is S, T, or A;
wherein X6 is R, T, H, D, E, N, M, or Q;
wherein X7 is L, G, A, IM, Y, or F; and
wherein X8 is G, R, A, V, L, or I

In one embodiment, the nSURF fragment comprises a polypeptide having at least 90%, at least 95% or at least 99% sequence identity to SEQ ID NO: 5:

VLQKFVGTWKIADSHNFGEYLRAIGSPKELSDGGDATRPTLYISQ
KDGDKMTVKIENGPPTFLDTQVKLKLGEEFDEFPSDGR.

In one embodiment, the nSURF fragment comprises a polypeptide having at least 90%, at least 95% or at least 99% sequence identity to SEQ ID NO: 6:

VLQKFVGTWKIADSHNFGEYLRAIGAPKELSDGGDATTPTLYISQ
KDGDKMTVKIENGPPTFLDTQVKFKLGEEFDEFPSDRR.

In a primary embodiment, the cSURF fragment comprises a polypeptide having at least 90%, at least 95% or at least 99% sequence identity to amino acids 85-139 of SEQ ID NO: 1 or SEQ ID NO: 2 or SEQ ID NO: 3. In one embodiment, the cSURF fragment comprises a polypeptide having at least 90%, at least 95% or at least 99% sequence identity to SEQ ID NO: 7:

GVKSVVNLVGEKLVYVQKWDGKETTX₁VREIKDGKLVVTLTMGDV
VAVRSYRRATE,

wherein X1 is H, D, E, N, M, or Q. In one embodiment, the cSURF fragment comprises a polypeptide having at least 90%, at least 95% or at least 99% sequence identity to SEQ ID NO: 8:

GVKSVVNLVGEKLVYVQKWDGKETTHVREIKDGKLVVTLTMGDVV
AVRSYRRATE.

In alternative implementations, the BiFC assay of the invention may comprise three or more fragments of the engineered UnaG protein of the invention, enabling the detection of PPIs encompassing three or more proteins. In such implementations, additional split sites are created to create the three or more fragments of UnaG.

### C. BiFC Assays.

The scope of the invention encompasses novel BiFC assays utilizing the complementary fragments of the engineered UnaG protein as disclosed above. The BiFC assays of the invention comprise two complementary constructs, comprising:
a first BiFC construct comprising a first fluorescent protein fragment, comprising a fragment of an engineered UnaG protein, joined to a first interacting partner; and
a second BiFC construct comprising a second fluorescent protein fragment, comprising a fragment of the engineered UnaG protein, joined to a second interacting partner;
wherein the engineered UnaG protein comprises a sequence having at least 90%, at least 95%, or at least 99% sequence identity to SEQ ID NO: 1, SEQ ID NO: 2, or SEQ ID NO: 3;
wherein, interaction of the first and second interacting partners brings the first and second fluorescent protein fragments into sufficient proximity to generate a fluorescent signal in response to illumination with a suitable wavelength of light.

In a primary embodiment, the first and second interacting partners are proteins. In this implementation, each of the first and second interacting partners is a PPI partner. In this implementation, each BiFC construct may conveniently be configured as a fusion protein wherein the fluorescent protein fragment and PPI partners are elements of a common amino acid sequence. In alternative implementations, the PPI Partner and engineered UnaG fragment are otherwise joined, for example, by conjugation chemistry.

Each complementary construct of the BiFC assays of the invention will comprise a fluorescent protein fragment joined (e.g., fused) to a selected interacting partner. Additionally, a linker sequence, as described below, may optionally be present between the fluorescent protein fragment and the interacting partner. The linker will provide steric flexibility to enable better interaction between interacting species and to facilitate reconstitution of the chromophore by the fluorescent protein fragments, and to avoid interference of the fluorescent protein fragments with the interaction. The linker may comprise any chemical species, typically a polymeric species. In each BiFC construct, the arrangement of the fluorescent protein and interacting species may be selected to optimize the interaction and chromophore reconstitution, i.e. the fluorescent protein may be joined to the interacting partner at various sites and the site which maximizes signal may be selected. For example, in the case of a BiFC construct comprising a fusion protein, the arrangement of the elements may be:

N-terminus−[Engineered UnaG Fragment]−[Linker]−[PPI Partner]−C terminus; or

N-terminus−[PPI Partner]−[Linker]−[Engineered UnaG fragment]−C-terminus.

The arrangement of the elements in the construct may be selected by one of skill in the art to minimize interference with the PPI by fusing at the end of the PPI partner that is less likely to be involved in the PPI. This may be determined in each case by the topography of the interacting domains on each PPI partner.

The BiFC constructs may optionally comprise a linker sequence between the UnaG fragment and the PPI Partner to which it is fused. In a primary implementation, the linker is an amino acid sequence. The linker may comprise an amino acid sequence of any length, for example, a sequence having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30 to 40, 40 to 50, 50 to 60, 60 to 70, 70 to 80, 80-90, 90-100, or an amount of over 100 amino acids. Exemplary linkers are in the range of 2-20 amino acids, for example 10-15 amino acids. In some implementations, the linker comprises one, two, or three amino acids, for example the products of restriction enzyme cut sites in the parent nucleic acid sequence. In one implementation, the linker will be a biologically inactive polypeptide and will be flexible. Exemplary linker sequences comprise glycine, alanine, and/or serine rich sequences or combinations thereof, for example, sequences comprising at least 50% glycine, alanine, and/or serine, at least 75% glycine, alanine, or and/or serine, or at least 90% glycine and/or serine. In one embodiment, the linker comprises one or more linker sequences of SEQ ID NO: 9: GGSA, for example, 1, 2, 3, 4, 5, 6, 7, 8, 9, 10, 11, 12, 13, 14, 15, 16, 17, 18, 19, or 20 repeats of GGSA.

The PPI partners may comprise interacting partners from any known PPI, or may comprise putative interacting proteins. Exemplary PPI partners include, for example, ligands and receptors, transcription factors, pathogen proteins and host targets, partners in a signal transduction network, enzymes and substrates, and nucleic acids. The PPI partners may comprise entire proteins or may comprise interacting motifs thereof. Interacting motifs thereof may include, for example, binding domains, binding sites for opposing PPI partners, and other elements that facilitate the selected PPI. For example, as described in the Examples herein, various PPIs were detected and measured, encompassing a variety of different proteins and interacting portions thereof, in order to demonstrate the versatility and general applicability of the detection systems disclosed herein to measure diverse PPIs. Examples of complementary PPI partners demonstrated herein include: PAR1 and beta-arrestin; p53 and Mdm2 (for example, amino acids 1-81 of p53 and amino acids 11-119 of Mdm2); KRas^{G12V} and Raf1 (for example, full length KRas and amino acids 50-131 of Raf1); YAP and TEAD (for example, full length YAP1 isoform 2 and full length TEAD); and FKBP and FRB (for example, full length FKBP1A and mTOR FRB, comprising amino acids 2025-2114).

Although the primary implementation of the invention is directed to protein-protein interactions, the scope of the invention extends to BiFC assays for detecting other types of interactions between two or more interacting species. For example, in one implementation the two interacting species are non-protein species. In another implementation, one interaction partner may comprise a protein while the other is a non-protein. For example, in one embodiment, the interacting partners are a protein, such as a DNA-binding protein, and a nucleic acid sequence with which the selected protein interacts. Non-protein PPI Partners may include, for example, nucleic acids, small molecules, carbohydrates, lipid molecules, and other species with which a selected protein interacts. In the case of non-protein interacting partners, as these cannot be expressed as a fusion partner with the engineered UnaG fragment of the construct, the interacting species generally must be conjugated to the engineered UnaG fragment by conjugation chemistries known in the art. For example, nucleic acid sequences and others may be conjugated with MUnaG protein fragments by any number of tools, including digoxigenin-modified nucleic acid sequences bound by digoxigenin-binding antibodies or antibody fragments fused to the selected MUnaG fragment, biotin-avidin functionalized nucleic acid and proteins, click chemistry moieties, for example, the use of azide-modified engineered UnaG fragment bound to DBCO-functionalized target, such as a nucleic acid sequence. In one embodiment, amine groups on the engineered UnaG protein fragment are used as attachment sites for thiolated nucleic acids.

### C. Nucleic Acid Constructs

The scope of the invention encompasses nucleic acid sequences which code for the selected engineered UnaG protein, selected UnaG protein fragment, and BiFC constructs of the invention. By the degeneracy of the genetic code, and based on the codon preferences of the selected expression system expressing the target proteins, one of skill in the art may readily derive appropriate nucleic acid sequences which code for the selected protein, fragment, or BiFC construct described above.

In one embodiment, the scope of the invention encompasses a nucleic acid sequence coding for an engineered UnaG protein of the invention, for example, a nucleic acid construct coding for a protein having at least 90%, at least 95%, or at least 99% sequence identity to a protein comprising SEQ ID NO: 1, SEQ ID NO: 2, or SEQ ID NO: 3.

In one embodiment, the scope of the invention encompasses a nucleic acid sequence coding for one or both complementary fragments of an engineered UnaG protein. In one embodiment, the nucleic acid sequence codes for a protein having at least 90%, at least 95%, or at least 99% sequence identity to SEQ ID NO: 4, SEQ ID NO: 5, SEQ ID NO: 6, SEQ ID NO: 7, or SEQ ID NO: 8. For example, in one embodiment the nucleic acid sequence codes for an n-terminal fragment of the engineered UnaG of SEQ ID NO: 6, for example, comprising a nucleic acid sequence having at least 90%, at least 95%, or at least 99% sequence identity to SEQ ID NO: 10:

gtgctccagaaattcgtaggaacttggaagatagcagattcacat
aatttcggcgaatatctcagagccataggagcaccaaaggaatta
tcagatggcggtgatgccacaactcccactctgtatatcagccag
aaagatggcgacaaaatgacggtgaaaatagagaacggcccaccg
accttcctggacactcaggtgaagtttaaactgggtgaggagttt
gacgagtttccttctgacgggcgt.

In one embodiment the nucleic acid sequence codes for the c-terminal fragment of engineered UnaG SEQ ID NO: 8, for example, comprising a nucleic acid sequence having at least 90%, at least 95%, or at least 99% sequence identity to SEQ ID NO: 11:

ggtgttaaaagcgtcgttaacctggtgggagagaaattagtatac
gtccaaaagtgggacggcaaggagactacgcacgttagagagatt
aaagacggcaagctggtggtaacactaacaatgggcgacgtcgtt
gcagtgcgctcatatcgcagggcgacggag.

In one embodiment, the nucleic acid sequence of the invention encompasses a nucleic acid sequence coding for a BiFC construct comprising an engineered UnaG fragment and a PPI Partner comprising a polypeptide or protein. In one embodiment, the nucleic acid sequence encodes two complementary BiFC constructs in a single sequence, wherein the BiFC constructs are separated by an intervening “self-cleaving” peptide sequence, as known in the art. For example, the self-cleaving peptide sequence may comprise a 2A sequence, for example, a 2A sequence selected from the group consisting of P2A, F2A, T2A, or E2A. Upon translation of the single mRNA coding for both BiFC construct monomers, the self-cleavable moiety splits the protein, resulting in the formation of two separate BiFC construct monomer fusion proteins. The use of such a construct enables expression of the reporter system from a single transformation event with a polycistronic vector. In the typical implementation, the monomers are expressed in a 1:1 ratio, however different stoichiometries may be used.

The nucleic acid sequences of the invention may encompass any form and format. For example, the nucleic acid sequences may comprise DNA, RNA, or other nucleotides or mixtures thereof. The nucleic acid sequences may comprise plasmids, cloning vectors, transformation vectors, or sequences integrated into the genome of an organism. The nucleic acid sequences may be formatted for expression systems of any type, for example for use in cell-free protein synthesis systems, for transformation of bacterial, yeast, insect, mammalian, or plant cells, for example in cell culture. The nucleic acid sequences of the invention may be configured for transduction of multicellular organisms such as test animals and animal models. The nucleic acid sequences of the invention may codon-optimized for the selected expression system.

The nucleic acid sequences of the invention may comprise additional elements. For example, the nucleic acid sequences may express BiFC constructs in combination with other fluorescent proteins, for example, as controls, or to facilitate cell detection and delineation. The nucleic acid sequences may code for genes that impart or augment bilirubin formation in the target cell, for example, heme oxygenase-1 (HO1) and biliverdin reductase (BvdR) for producing bilirubin in E. coli or other bacterial systems.

### D. PPI Detection Systems

The scope of the invention encompasses PPI detection systems. The PPI detection system will comprise two complementary BiFC constructs, each comprising a complementary fragment of an engineered UnaG protein of the invention, e.g. complementary fragments of SEQ ID NO: 1, SEQ ID NO: 2 or SEQ ID NO: 3, for example, fragments comprising SEQ ID NO: 4, SEQ ID NO: 5, SEQ ID NO: 6, SEQ ID NO: 7, or SEQ ID NO: 8. The system will comprise an environment in which the PPI of the PPI partners of the BiFC will interact, or wherein they may be induced to interact. In a primary embodiment, the PPI detection system comprises a cell, wherein the cell expresses two complementary BiFC constructs, each comprising a complementary fragment of an engineered UnaG protein of the invention, e.g. complementary fragments of SEQ ID NO: 1, SEQ ID NO: 2, or SEQ ID NO: 3, for example, fragments comprising SEQ ID NO: 4, SEQ ID NO: 5, SEQ ID NO: 6, SEQ ID NO: 7, or SEQ ID NO: 8. In various embodiments, cell may comprise any of: a bacterial cell, a yeast cell, an insect cell, an animal cell, a mammalian cell, or a plant cell. Exemplary human cell lines include HEK293 cells, HeLa cells, Jurkat cells, PC3 cells and other human cell lines known in the art. Exemplary cell lines further include CHO cells, Sf9 cells, E. coli cells, Ns0, and Sp2/0 cells. The cells may comprise cultured cells, such as cells on a culture substrate or suspension culture. The cells may comprise cells in the tissue or organ of a living organism, such as a test animal. The cells may comprise cells in an organoid. The cells may be cultured or imaged in a vessel, such as a well of a multiwell plate, for example, to enable high throughput screening.

Expression of the two complementary BiFC constructs in the cell may be achieved by any methodology known in the art, for example, by viral vector (e.g. adenovirus or adeno-associated virus, lentivirus), nanoparticle mediated gene delivery (e.g. dendrimers, lipids, chitosan gene delivery particles, etc.), electroporation, biolistic delivery systems, microinjection, ultrasound, hydrodynamic delivery, liposomal delivery, extracellular vesicle-mediated delivery (e.g. exosome, nanovesicle), polymeric or protein-based cationic agents (e.g. polyethylene imine, polylysine), intraject systems, and DNA-delivery dendrimers. The expression may be stable or transient.

In an alternative implementation, the PPI detection system is an in vitro system, for example, a vessel, well, or other containment, containing a medium, such as buffer or growth medium, into which the two complementary BiFC constructs may be introduced under conditions wherein the selected PPI occurs or may be induced.

In one embodiment, the scope of the invention encompasses a BiFC assay for the detection of a selected PPI, the BiFC assay comprising
a cell,
   wherein the cell is engineered to express a first and a second BiFC construct, wherein
      the first BiFC construct comprises a first PPI partner of a selected PPI fused to a first fluorescent protein fragment; and
      the second BiFC construct comprises a second PPI partner of the selected interaction fused to a second fluorescent protein fragment;
      wherein the first and second fluorescent fragments comprise complementary fragments of an engineered UnaG protein, the engineered UnaG protein
      comprising a protein having at least 90%, at least 95% or at least 99% sequence identity to SEQ ID NO: 1, SEQ ID NO: 2, or SEQ ID NO: 3.

In various embodiments:
the first fluorescent protein fragment comprises amino acids 1-70 of the protein having at least 90%, at least 95% or at least 99% sequence identity to SEQ ID NO: 1, SEQ ID NO: 2, or SEQ ID NO: 3 and the second fluorescent protein fragment comprises amino acids 90-139 of the protein having at least 90%, at least 95% or at least 99% sequence identity to SEQ ID NO: 1, SEQ ID NO: 2, or SEQ ID NO: 3;
the first fluorescent protein fragment comprises amino acids 1-83 of the protein having at least 90%, at least 95% or at least 99% sequence identity to SEQ ID NO: 1, SEQ ID NO: 2, or SEQ ID NO: 3 and the second fluorescent protein fragment comprises amino acids 85-139 of the protein having at least 90%, at least 95% or at least 99% sequence identity to SEQ ID NO: 1, SEQ ID NO: 2, or SEQ ID NO: 3;
the first fluorescent protein fragment comprises SEQ ID NO: 4 and the second fluorescent protein fragment comprises SEQ ID NO: 7;
the first fluorescent protein fragment comprises SEQ ID NO: 5 and the second fluorescent protein fragment comprises SEQ ID NO: 8;
the first fluorescent protein fragment comprises SEQ ID NO: 6 and the second fluorescent protein fragment comprises SEQ ID NO: 8;
in the first BiFC construct a linker sequence is present between the first PPI partner and the first fluorescent protein fragment and/or in the second BiFC construct a linker sequence is present between the second PPI partner and the second fluorescent protein fragment, and/or the linker may comprise SEQ ID NO: 9 or repeats thereof;
the cell comprises any of a bacterial cell, a yeast cell, an insect cell, an animal cell, a mammalian cell, or a plant cell, a HEK293 cell, a HeLa cell, a Jurkat cell, a PC3 cell, a cell of a cancer cell line, a CHO cell, an Sf9 cell, an E. coli cell, an Ns0 cell, an Sp2/0 cell; a cultured cell, a cell present in a tissue or organ of a living organism or an explant thereof, and a cell in an organoid;
and/or
the cell expresses a nucleic acid sequence comprising SEQ ID NO: 10, a nucleic acid sequence comprising SEQ ID NO: 11, or a nucleic acid sequence comprising SEQ ID NO: 10 and SEQ ID NO: 11.

In one embodiment, the BiFC assay of the invention is configured to detect the PPI between KRas^{G12V} and Raf1. In such implementation, the first PPI partner comprises a protein comprising KRas^{G12V} or a subsequence thereof which interacts with Raf1; the second PPI partner comprises Raf1 or a subsequence thereof which interacts with KRas^{G12V}, for example, the receptor binding domain of Raf1. In one embodiment, BiFC assay of the invention comprises a first construct comprising KRas^{G12V} or a subsequence thereof which interacts with Raf1 fused to a first fluorescent protein fragment; and a second BiFC construct comprising Raf1 or a subsequence thereof which interacts with KRas^{G12V} fused to a second fluorescent protein fragment; wherein the first and second fluorescent fragments are complementary fragments of an engineered UnaG protein comprising a sequence having at least 90%, at least 95%, or at least 99% sequence identity to SEQ ID NO: 1, SEQ ID NO: 2, or SEQ ID NO: 3. In one embodiment, the first fluorescent protein fragment comprises a sequence having at least 90%, at least 95%, or at least 99% sequence identity to SEQ ID NO: 4, SEQ ID NO: 5, SEQ ID NO: 6, SEQ ID NO: 7, or SEQ ID NO: 8. In one embodiment, the second fluorescent protein fragment comprises a sequence having at least 90%, at least 95%, or at least 99% sequence identity to SEQ ID NO: 4, SEQ ID NO: 5, SEQ ID NO: 6, SEQ ID NO: 7, or SEQ ID NO: 8.

In one embodiment, the BiFC assay of the invention is configured to detect the PPI between YAP1 and TEAD. In such implementation, the first PPI partner comprises a protein comprising YAP1 or a subsequence thereof which interacts with TEAD; the second PPI partner comprises TEAD or a subsequence thereof which interacts with YAP1. In one embodiment, BiFC assay of the invention comprises a first construct comprising YAP1 or a subsequence thereof which interacts with TEAD fused to a first fluorescent protein fragment; and a second BiFC construct comprising TEAD or a subsequence thereof which interacts with YAP1 fused to a second fluorescent protein fragment; wherein the first and second fluorescent fragments are complementary fragments of an engineered UnaG protein comprising a sequence having at least 90%, at least 95%, or at least 99% sequence identity to SEQ ID NO: 1, SEQ ID NO: 2, or SEQ ID NO: 3. In one embodiment, the first fluorescent protein fragment comprises a sequence having at least 90%, at least 95%, or at least 99% sequence identity to SEQ ID NO: 4, SEQ ID NO: 5, SEQ ID NO: 6, SEQ ID NO: 7, or SEQ ID NO: 8. In one embodiment, the second fluorescent protein fragment comprises a sequence having at least 90%, at least 95%, or at least 99% sequence identity to SEQ ID NO: 4, SEQ ID NO: 5, SEQ ID NO: 6, SEQ ID NO: 7, or SEQ ID NO: 8.

In one embodiment, the BiFC assay of the invention is configured to detect the PPI between MYCN and AURKA. In such implementation, the first PPI partner comprises a protein comprising MYCN or a subsequence thereof which interacts with AURKA, for example, the N-terminal fragment of MYCN (e.g., amino acids 1-137 of MYCN); the second PPI partner comprises AURKA or a subsequence thereof which interacts with MYCN, for example, the AURKA kinase domain (e.g., amino acids 122-403 of AURKA). In one embodiment, BiFC assay of the invention comprises a first construct comprising MYCN or a subsequence thereof which interacts with AURKA fused to a first fluorescent protein fragment; and a second BiFC construct comprising AURKA or a subsequence thereof which interacts with MYCN fused to a second fluorescent protein fragment; wherein the first and second fluorescent fragments are complementary fragments of an engineered UnaG protein comprising a sequence having at least 90%, at least 95%, or at least 99% sequence identity to SEQ ID NO: 1, SEQ ID NO: 2, or SEQ ID NO: 3. In one embodiment, the first fluorescent protein fragment comprises a sequence having at least 90%, at least 95%, or at least 99% sequence identity to SEQ ID NO: 4, SEQ ID NO: 5, SEQ ID NO: 6, SEQ ID NO: 7, or SEQ ID NO: 8. In one embodiment, the second fluorescent protein fragment comprises a sequence having at least 90%, at least 95%, or at least 99% sequence identity to SEQ ID NO: 4, SEQ ID NO: 5, SEQ ID NO: 6, SEQ ID NO: 7, or SEQ ID NO: 8.

In one embodiment, the BiFC assay of the invention is configured to detect the PPI between p53 and Mdm2. In such implementation, the first PPI partner comprises a protein comprising p53 or a subsequence thereof which interacts with Mdm2, for example, the amino acids 1-83 of p53; the second PPI partner comprises Mdm2 or a subsequence thereof which interacts with p53, for example, the amino acids 11-119 of Mdm2. In one embodiment, BiFC assay of the invention comprises a first construct comprising p53 or a subsequence thereof which interacts with Mdm2 fused to a first fluorescent protein fragment; and a second BiFC construct comprising Mdm2 or a subsequence thereof which interacts with p53 fused to a second fluorescent protein fragment; wherein the first and second fluorescent fragments are complementary fragments of an engineered UnaG protein comprising a sequence having at least 90%, at least 95%, or at least 99% sequence identity to SEQ ID NO: 1, SEQ ID NO: 2, or SEQ ID NO: 3. In one embodiment, the first fluorescent protein fragment comprises a sequence having at least 90%, at least 95%, or at least 99% sequence identity to SEQ ID NO: 4, SEQ ID NO: 5, SEQ ID NO: 6, SEQ ID NO: 7, or SEQ ID NO: 8. In one embodiment, the second fluorescent protein fragment comprises a sequence having at least 90%, at least 95%, or at least 99% sequence identity to SEQ ID NO: 4, SEQ ID NO: 5, SEQ ID NO: 6, SEQ ID NO: 7, or SEQ ID NO: 8.

### E. Methods of Use.

The improved UnaG mutants of the invention may be used in any context wherein fluorescent proteins are used. For example, improved UnaG proteins comprising SEQ ID NO: 1, SEQ ID NO: 2, SEQ ID NO: 3, or subsequences thereof may be used as reporters, for example, being expressed in cells, for example. In one embodiment, the improved UnaG proteins of the invention are expressed as fusion proteins with a protein of interest, a trafficking or localization signal, or other elements as known in the art.

In a primary implementation, the improved UnaG proteins of the invention are utilized in BiFC assays. The scope of the invention further encompasses a general method as follows:

A method of detecting a PPI between two interacting partners, the method comprising
introducing:
   a first BiFC construct, the first BiFC construct comprising a first interacting partner of a selected interaction joined to a first fluorescent protein fragment; and
   a second BiFC construct, the second BiFC construct comprising a second interacting partner of the selected interaction joined to a second fluorescent protein fragment;
   wherein the first and second fluorescent fragments comprise complementary fragments of an engineered UnaG protein, the engineered UnaG protein comprising a protein having at least 90%, at least 95% or at least 99% sequence identity to SEQ ID NO: 1, SEQ ID NO: 2, or SEQ ID NO: 3;
wherein, the first and second constructs are introduced under conditions suitable for the selected interaction to occur;
wherein, upon interaction of the first and second interacting species, the fluorescent protein fragments of the first and second BiFC constructs are brought into sufficient proximity to produce fluorescent signal when illuminated with energy of a suitable wavelength;
illuminating the first and second BiFC constructs with energy of the suitable wavelength and
by the use of a suitable detection system, detecting the fluorescent signal, if any, generated by the complementary fluorescent protein fragments.

The general method may be performed in various implementations and contexts. In one embodiment, the complementary fluorescent protein fragments comprise a polypeptide comprising at least 90%, at least 95%, or at least 99% sequence identity to SEQ ID NO: 4 and SEQ ID NO: 5, or SEQ ID NO: 6, SEQ ID NO: 7, or SEQ ID NO: 8. In one embodiment, the interacting partners are proteins or interacting fragments or domains thereof. In one embodiment, the introduction of the complementary BiFC constructs is achieved by their expression in a cell.

Conditions suitable for the PPI to occur may be steady state conditions of the cell, or may be induced by application of activating species. For example, in the case of FKBP/Fbr PPI detection system, introduction of rapamycin will induce the PPI. Bilirubin is necessary for generation of UnaG fluorescence. In many cell types, endogenous bilirubin is sufficiently abundant to enable signal generation If bilirubin is not present within the cells or is present in limiting concentrations, it may be applied exogenously. Alternatively, the cells may be engineered to express enzymes that to produce bilirubin. For example, in E. coli, the expression of heme oxygenase-1 (HO1) and biliverdin reductase (BvdR) will result in endogenous production of bilirubin to ensure fluorescence. The bilirubin producing enzymes may be co-expressed with the BiFC constructs in the target cells.

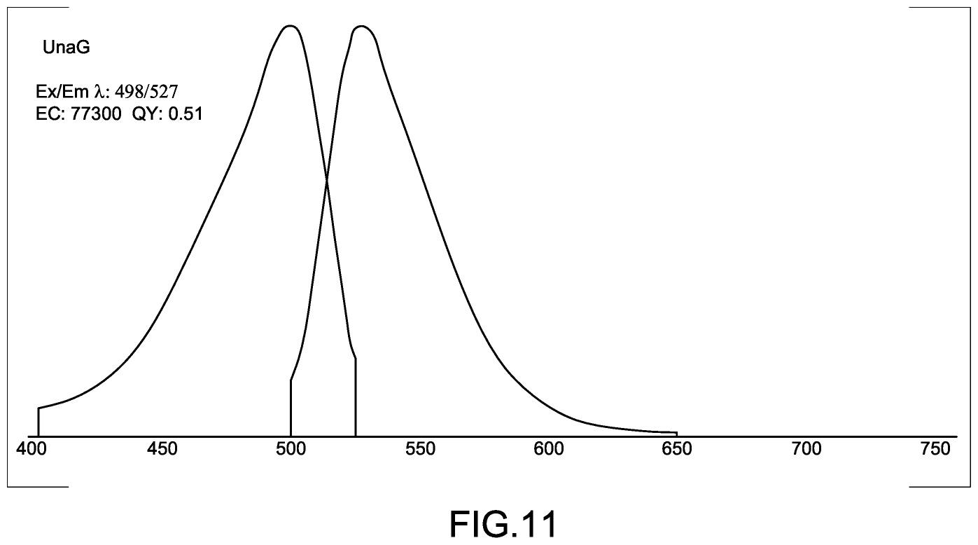

Fluorescent protein signals may be analyzed with techniques known in the art. Quantitative or qualitative measurement may be performed. Imaging of fluorescent proteins is readily accomplished with a variety of techniques, including, but not limited to widefield, confocal, 2P, multiphoton microscopy, wide-confocal laser scanning microscopy, live-cell time-lapse fluorescence confocal microscopy, and others known in the art. The detection system will comprise elements, e.g. lasers, for illumination at wavelengths that induce signal, for example, at wavelengths of 450-500 nm, with peak excitation observed at 498 nm. The detection system will further comprise elements for detection of engineered UnaG signals, for example, at wavelengths of 500-580 nm, with peak emission observed at 527 nm, as depicted in FIG. 11. For example, in an exemplary implementation, fluorescence imaging may be performed by an inverted microscope equipped with a confocal scanner unit, a digital CMOS camera, an automated stage, laser inputs with laser lines at about 498 nm for UnaG imaging, and an emission filters of 525/50-nm for UnaG imaging. With bright signal, in one embodiment the system provides a facile qualitative analysis based on raw images, without the need for additional quantitative data analysis. Alternatively, the system may act as a quantitative indicator of the selected-protein protein interaction, as the abundance of fluorescent signals are proportional to the scale of the protein-protein interaction. Signal may be quantified by any appropriate technique. In one embodiment, the sum of fluorescent droplets' pixel intensity divided by the sum of the cell's overall pixel intensity is utilized as the measure of signal. Bulk measurement of fluorescent signal in a selected assay may be used, for example, fluorescent signal from the well of a culture dish comprising cells expressing complementary BiFC constructs, fluorescent signal from one or more cells expression complementary BiFC constructs, or fluorescent signal from selected areas of one or more cells expressing complementary BiFC constructs. In one implementation, signal is measured in a scan taken along a selected line across the width of a cell. In some cases, based on the site of the interaction, signal will be present in localized islands or areas of signal. In one implementation, a histograms of signal intensity across a cell may be generated, wherein the area under the line is quantitative for signal, for example, as presented in FIG. 2. Depending on the site of the PPI in the cell, the signal may be localized to part of the cell, e.g. the cell membrane or the nucleus, or may be evenly distributed throughout the cell. Relevant sections of the cell may be assessed accordingly. Fluorescence can be normalized against expression of one or more control reporters, for example, co-expressed fluorescent proteins that generate signals which are not affected by the PPI. Exemplary proteins include RFP, YFP, and mCherry.

In one implementation, a representative number of fields of view (FOV), are selected from the assay, for example, 3-20 FOVs per well of cultured cells expressing the BiFC system of the invention, for example, 10 FOVs. In one embodiment, the following are measured: SURF fluorescence per one FOV; the fluorescence of a co-expressed control fluorescent protein (e.g. mCherry) per FOV, from which SURF fluorescence normalized by the control fluorescent protein (e.g. mCherry) may be calculated. When assessing putative inhibitors or activators of the PPI, the fold change of normalized SURF fluorescence in response to the addition of the agent can be assessed by techniques known in the art.

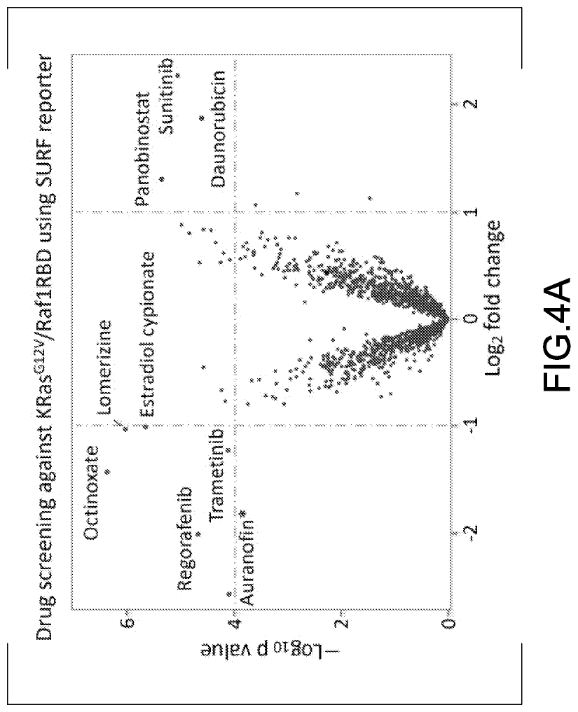

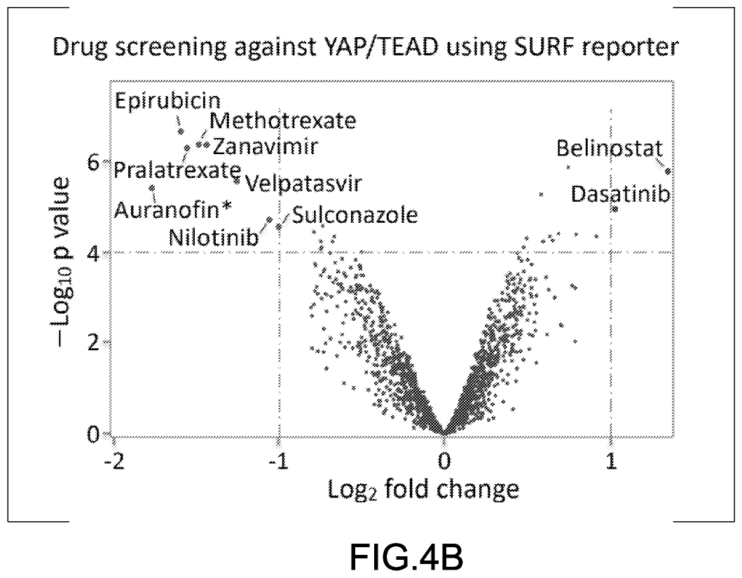

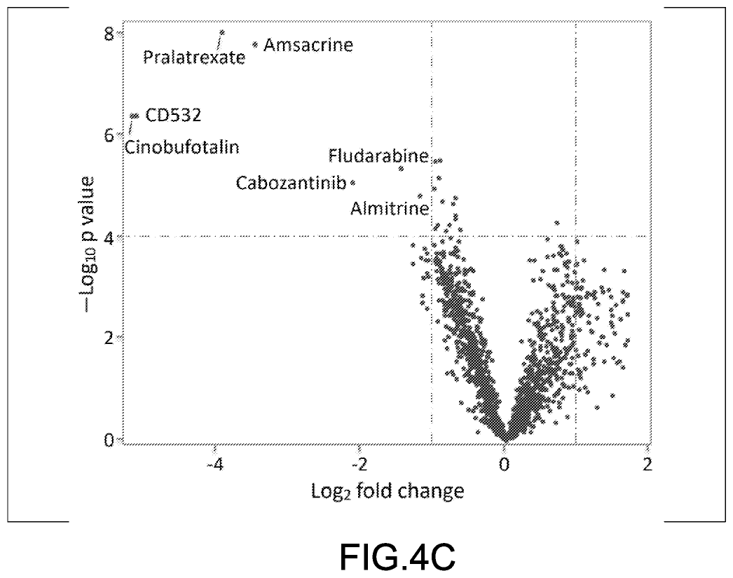

In the method of the invention, PPIs may be measured in any number of contexts. Exemplary uses include confirming putative PPIs, screening and identifying modulators of PPIs, quantifying PPIs in response to activators on inhibitors, and other uses of BiFC assays known in the art. For example, as described herein in the examples, compositions comprising putative inhibitors of the selected PPI may be introduced to the PPI detection systems of the invention. If a reduction in signal, relative to untreated systems (e.g. cells) is achieved by the introduction of a composition, the composition is deemed to be an inhibitor of the PPI. Likewise, species that enhance PPI signal, relative to untreated systems (e.g. cells), the species is deemed to be an activator of the PPI. The systems of the invention are highly amenable to use in high-throughput screening systems, for example, comprising cells cultured in multiwell plates. For example, as described in the examples herein, high throughput screening of 1622 drugs was performed for three different PPI reporting systems, and inhibitors and enhancers of the selected PPIs were identified (FIGS. 4A, 4B, and 4C).

In one implementation, the scope of the invention encompasses a method of detecting modulation of an interaction between two interacting partners, the method comprising
introducing:
   a first BiFC construct, the first BiFC construct comprising a first interacting partner of a selected interaction joined to a first fluorescent protein fragment; and
   a second BiFC construct, the second BiFC construct comprising a second interacting partner of the selected interaction joined to a second fluorescent protein fragment;
   wherein the first and second fluorescent fragments comprise complementary fragments of an engineered UnaG protein, the engineered UnaG protein comprising a protein having at least 90%, at least 95% or at least 99% sequence identity to SEQ ID NO: 1, SEQ ID NO: 2, or SEQ ID NO: 3; and
   wherein, upon interaction of the first and second interacting species, the fluorescent protein fragments of the first and second BiFC constructs will be in sufficient proximity to produce fluorescent signal when illuminated with energy of a suitable wavelength;
wherein, the first and second constructs are introduced under conditions suitable for the selected interaction to occur;
wherein the selected agent is introduced to the first and second construct;
illuminating the first and second BiFC constructs with energy of the suitable wavelength
and
by the use of a suitable detection system, detecting the fluorescent signal, if any, generated by the complementary fluorescent protein fragments;
wherein, if the fluorescent signal generated is changed, compared to signal generated in like systems lacking the agent, the agent is deemed to a modulator of the interaction.

The foregoing method may be applied in various contexts. Suitable controls for assessing the effect of the selected modulator may include untreated BiFC assays run in parallel, or previously run BiFC assays using the same elements in the absence of the selected agent. In one embodiment, the interaction is a PPI and the interacting partners are proteins. In one embodiment, the BiFC assay comprises a cell wherein the first and second constructs comprise fusion proteins expressed therein. In one embodiment, the PPI is a PPI underlying a pathological condition, e.g. a proliferative condition, i.e. cancer or other neoplasm. In one embodiment, the selected agent is a putative inhibitor. In one embodiment, the selected agent is a putative enhancer. In one embodiment, the method is performed in parallel with a plurality of selected agents, as in a high throughput screen.

### F. EXAMPLES

### Example 1. Engineering SURF, a Reversible and Bright Protein-Protein Interaction Reporter

SURF is engineered from UnaG, a fluorescent protein cloned recently from the Japanese eel Unagi [Kumagai 2013]. UnaG incorporates an endogenous molecule bilirubin as the chromophore. Thus, its fluorescence is genetically encoded requiring no exogenous cofactor. Bilirubin is a tetrapyrrole bilin and free bilirubin is not fluorescent. Using a structure-guided approach an UnaG-based protein complementation assay was designed by splitting UnaG into two parts, for example, as described in To, 2016. Next, using directed evolution, the brightness was improved about ˜100-fold. Briefly, a construct, referred to herein as cpUnaG, was made comprising the previously engineered split UnaG: cUnaG (carboxyl-terminal fragment of UnaG from residues 85 to 139, i.e. UnaG⁸⁵⁻¹³⁹) and nUnaG (amino-terminal fragment of UnaG residues 1 to 84, i.e. UnaG¹⁻⁸⁴), wherein the complementary fragments were separated by a short linker of a few amino acids, assuming the configuration: [C-terminal fragment 85-139]-[linker]-[N-terminal fragment 1-84]. A mutant library of cpUnaG was generated by random mutagenesis via error-prone PCR. The library was cloned into a pBAD vector, in which two genes, heme oxygenase-1 (HO1) and biliverdin reductase (BvdR), were added into the pBAD vector for producing bilirubin in E. coli. The mutant library was expressed in E. coli, and brighter colonies were selected. To combine beneficial mutations of the improved mutants, DNA shuffling was used to create a new library. The brightest mutant was then selected and subjected to a second round of random mutagenesis. After five rounds of directed evolution, ˜100-fold brighter mutant was identified (FIG. 10). This mutant contained 5 mutations, with two of the mutations located near the chromophore: MIV, V2L, E3Q, K22R, and Y110H, SEQ ID NO: 3. Next, a nucleic acid sequences were made coding for BiFC constructs: the new cSURF (SEQ ID NO: 8) fused to FKBP and the new nSURF (SEQ ID NO: 6) fused to Frb. Expression of these constructs in HEK293 cells showed that in the presence of rapamycin, this evolved split UnaG mutant was ˜100-fold brighter in detecting the FKBP and Frb interaction than the original split UnaG. This significantly improved split UnaG mutant was named SURF (Split UnaG-based Reversible and Fluorogenic PPI reporter).

### EXAMPLE 2. BiFC Assays Utilizing SURF

Overview: The novel mutant UnaG protein of Example 1 was next utilized in BiFC assays. As demonstrated below, SURF showed several unique advantages to existing PPI reporters:
1) Large dynamic range (dark-to-bright fluorescence), enabling sensitive & robust imaging & screening of PPI inhibitors.
2) Reversible with fast kinetics (single-digit minute timescale), which enables rapid detection of PPI inhibition.
3) Genetically encoded requiring no exogenous cofactor for fluorescence, enabling live cell imaging with no perturbation.

In various experiments, SURF was visualize and detect PPIs of diverse protein families with spatiotemporal dynamics in cells, including G protein-coupled receptors, E3 ubiquitin ligase and substrates, small GTPases (e.g. KRas and Raf1), transcription factors (e.g. b-catenin and TCF4). SURF-based assays are bright and robust (Z′-factor≥0.8) and thus well suited for high throughput screening of PPI inhibitors against several oncoproteins. For example, in one implementation, a SURF assay for imaging PPI between MYCN and AURKA was made. In another implementation, a SURF assay for imaging the PPI between KRas^{G12V} and Raf1 was made. In another implementation, a SURF assay for imaging the PPI between YAP and TEAD was made.

In additional experiments, these SURF assays were used to screen an FDA-approved drug library, by which screen, several drugs that inhibit or enhance certain PPIs were identified. the interaction between MYCN and AURKA. Furthermore, three of the identified drugs have been validated to degrade MYCN proteins in the MYON-amplified neuroblastoma cells, and block cell proliferation and the blocking is proportional to MYCN expression levels in various neuroblastoma cell lines. We have also screened and identified drugs that inhibit the PPI between KRas^{G12V} and Raf1, and the PPI between YAP and TEAD, in cells.

MYC oncoproteins. Myc oncoprotein rely on interaction with several key proteins for activity and stability, and inhibition of these interactions results in MYC inactivation and/or degradation. First, MYC (including MYCN and c-MYC) interacts with MAX through a conserved domain at the carboxyl-terminal region of each protein, which encompasses a helix-loop-helix (HLH) domain and a leucine zipper (LZ). Heterodimerization of MYC/MAX places the HLH-adjacent basic region of each protein in a manner that leads the heterodimer to bind to a consensus sequence on DNA known as enhancer box (E-box) element. This results in transcriptional activation and expression of downstream target genes that reprogram diverse cellular processes including proliferation, differentiation, cell-cycle progression, metabolism, apoptosis, angiogenesis. Disruption of MYC/MAX heterodimer by a dominant negative MYC peptide, named Omomyc, inhibits MYC-binding to the E-box consensus recognition elements, blocks MYC-dependent transcriptional activation, impairs MYC-driven gene expression and reverses MYC-induced transformation in vitro and MYC-driven tumorigenesis in vivo 31. While small molecule inhibitors of MYC/MAX interaction have been developed very recently, they may show cellular toxicity, which limits their therapeutic index. Therefore, there is a need to identify new inhibitors with large therapeutic index against the PPI of MYC with MAX. Second, MYC (including MYCN, c-MYC)-mediated transcriptional activation requires interaction with a nuclear factor TRRAP (transformation/transcription domain-associated protein), via a conserved domain at the amino-terminal region of MYC, known as MYC Box II (MBII). TRRAP is in a complex with HATs (histone acetyltransferases) that acetylate histones, inducing an open chromatin conformation around gene promoters, recruiting RNA polymerase II, leading to productive transcription 20-22. Disruption of MYC/TRRAP interaction by a dominant negative TRRAP inhibits MYC activity and abolishes MYC-mediated oncogenic transformation. Therefore, identifying small molecule PPI inhibitors of MYC/TRRAP should lead to promising therapeutics. Lastly, MYC interacts with aurora kinase A (AURKA) in a kinase activity-independent manner, and inhibition of this interaction results in MYC degradation via the ubiquitin-proteasome system. In normal cells, MYC protein stability is highly regulated via post-translational modifications: 1) MYC proteins are phosphorylated by upstream kinases including GSK3 and CDK1; 2) phosphorylated MYC recruits the E3 ubiquitin ligase complex SCFFBXW7 via interacting with the F-box protein FBXW7, resulting in ubiquitination and degradation. In cancer cells, however, highly expressed MYC interacts with aurora A, which induces a conformational change of the MYC/FBXW7 complex so that MYC is no longer efficiently ubiquitinated by SCFFBXW7, leading to reduced proteasomal degradation, resulting in MYC stabilization 13.38. MYC interacts with aurora A via a conserved domain at the amino-terminal region of MYC, known as MYC Box I (MBI) 13. Disruption of MYC/aurora A interaction by an allosteric inhibitor CD532 leads to MYC degradation in the tumor cells

While PPI inhibition is a valid anti-cancer approach, no drugs have been approved against MYC oncoproteins. This is because protein interaction interface is often flat and has much larger area than traditional active site of an enzyme such as kinase, thus it is difficult to design a small molecule that can tightly bind to the interface for efficient competition and disruption of interacting proteins. While structure-based design of PPI inhibitors is challenging due to relatively large and flat interface, high throughput screening is an alternative and promising approach, which may identify inhibitors that bind to cryptic binding sites that are hidden in the static x-ray crystal structures of proteins. One challenge of this approach is lack of a robust imaging assay. For example, Förster resonance energy transfer (FRET)-based PPI assays have small dynamic range and thus are difficult to use, due to small fluorescence change of the donor and acceptor fluorophores. To overcome this, we the BiFC protein-fragment complementation assays disclosed herein provide a sensitive and robust PPI assay with orders of magnitude larger dynamic range than FRET.

To enable robust imaging of PPIs for high throughput screening (HTS) of PPI inhibitors in cellular context, a sensitive PPI reporter that is reversible and fluorogenic using protein-fragment complementation, also known as bimolecular fluorescence complementation was used: i) a fluorescent reporter is split into two parts, which are each fused to a protein of interest X and Y. When the two proteins interact, this will bring two fragments of the reporter into close proximity so that they reconstitute and fluoresce; ii) when the two proteins are dissociated by inhibitors, a reversible reporter will also dissociate into two fragments and lose fluorescence, in contrast to an irreversible reporter that will not dissociate & no fluorescence change, and thus cannot detect PPI inhibition.

Results: The studies disclosed herein demonstrate that: 1) SURF can visualize and detect PPIs of diverse protein families with spatiotemporal dynamics in live cells, including G protein-coupled receptors, E3 ubiquitin ligase and substrates, small GTPases (e.g. KRas and Raf1), transcription factors (e.g. b-catenin and TCF4); 2) SURF-based assays enable high throughput screening of PPI inhibitors. For example, the SURF-based assay of MYCN and AURKA interaction can be used for high throughput screening. This SURF-based screening of 1622 FDA drugs identified six drugs that block this PPI. The inhibition kinetics of these drugs in living cells was then examined using the SURF assay, and five of them showed various degrees of inhibition within 6 hours. Among these, cinobufotalin showed the fastest inhibition kinetics with half-to-maximum time (T_{1/2}^{off}) ˜2 hours, which is similar to that of the compound CD532. Cabozantinib and amsacrine showed relatively fast kinetics with T_{1/2}^{off} ˜4 and 6 hours, respectively. The other two drugs showed the slowest inhibition kinetics with T_{1/2}^{off}>6 hours. Next, these drugs' effects on MYCN degradation in the MYON-amplified neuroblastoma cells (Kelly cells) was assessed using western blot analysis, and three of them showed significant degradation. Among these, cinobufotalin showed the strongest effect on MYCN degradation (similar to CD532), which is consistent with the fast inhibition kinetics. Lastly, all the three drugs suppressed proliferation of the neuroblastoma cells, and the degree of anti-proliferation is proportional to the MYCN amplification and expression levels. Second, SURF assays for KRas^{G12V} and Raf1; YAP and TEAD were also developed. FDA-approved drug library were screened with these assays, by which drugs that inhibit the PPI between KRas^{G12V} and Raf1; YAP and TEAD.

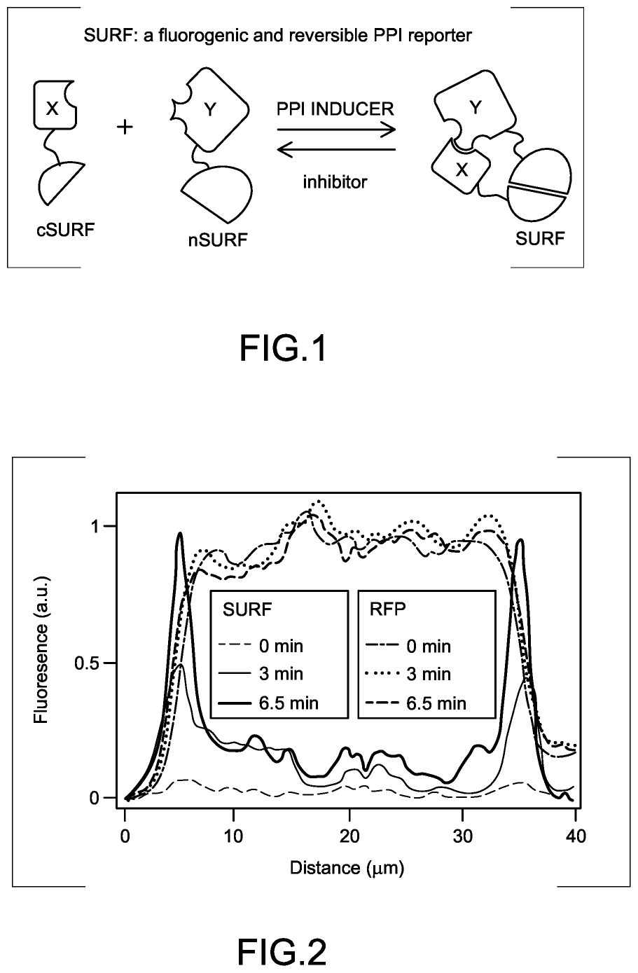

SURF visualized PPIs of diverse protein families. To visualize two proteins' interaction, each fragment of SURF (cSURF: carboxyl-terminal fragment; nSURF: amino-terminal fragment) was fused to a PPI partner of interest. When the two proteins interact, this brings the two fragments into close proximity so that SURF reconstitutes and becomes fluorescent (FIG. 1). PPI inhibition dissociates two interacting proteins, resulting in dissociation of the two SURF fragments and loss of fluorescence.

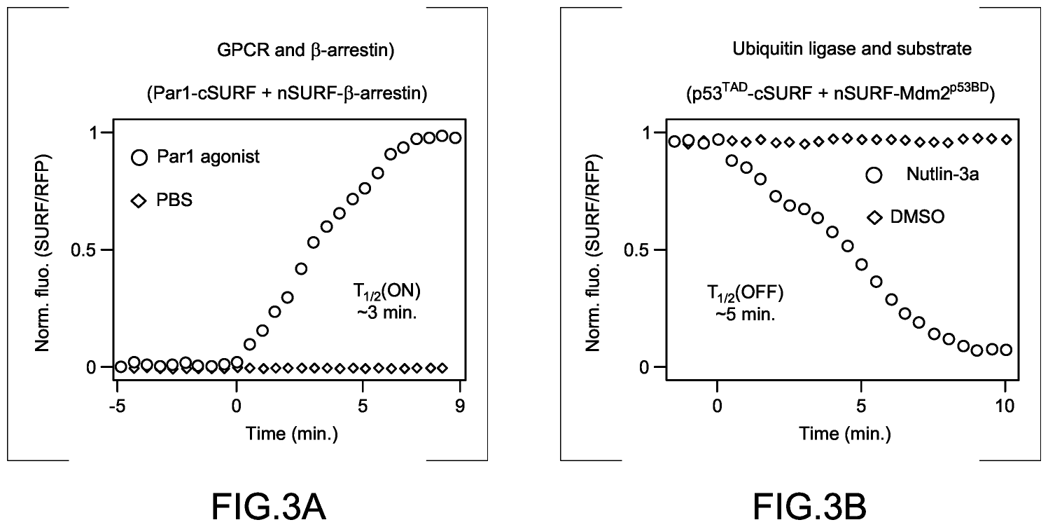

SURF visualized interaction between a G protein-coupled receptor (GPCR) and beta-arrestin. One fragment of SURF was fused to protease activated receptor-1 (PAR1); and the other fragment was fused to beta-arrestin. Addition of the PAR1 agonist activated this GPCR, promoting its phosphorylation, resulting in its interaction with beta-arrestin. Indeed, SURF fluorescence was detected on the plasma membrane upon addition of the PAR1 agonist (FIG. 2). Meanwhile, co-expressed RFP (red fluorescent protein) fluorescence was stable (FIG. 2). Quantitative analysis showed that the ON kinetics of SURF was ˜ 3 minutes (FIG. 3A), which is 10 times faster than known split GFP-based PPI reporters, which can take up to 30 minutes, due to the chromophore maturation step.

SURF visualized interactions between E3 ubiquitin ligase and its substrate. SURF visualized interactions between p53 and Mdm2, as well as Nutlin-3a induced dissociation, with OFF kinetics ˜5 minutes (FIG. 3B).

SURF visualized interactions between a small GTPase and its effector, including KRas and its effector Raf1 (i.e. C-Raf), Rho family small GTPases and their effectors such as RhoA, Cdc42 and Rac1, and other small GTPases and their effectors such as Ran and Ras-like protein (Rap1). GTP-bound small GTPases interact with their corresponding effector proteins, whereas GDP-bound small GTPases do not.

While Ras mutations are found in many human tumors, there is no FDA-approved Ras inhibitor in the clinic. Recently, ARS-853 (and ARS-1620) was developed to inhibit the KRas G12C mutant by stabilizing GDP-bound form and thus decreasing proportion of GTP-bound proteins. Indeed, SURF visualized fluorescence loss upon addition of ARS-853 for this mutant. And the kinetics of SURF fluorescence decrease is consistent with the inhibition mechanism of ARS-853, that requires several hours. Furthermore,

$${Z}^{′} - {factor}^{48} = 1 - \frac{( 3 {σ}_{c +} + 3 {σ}_{c -} )}{\vert {μ}_{c +} - {μ}_{c -} \vert} ,$$

was calculated as >0.8 at both 5 hrs and 24 hrs end points. ARS-853 does not affect the wild type KRas since its effect requires covalent binding to the cysteine at residue 12. Indeed, the SURF fluorescence of wild type KRas with Raf1 did not change upon addition of ARS-853. Thus, SURF visualized the interaction between KRas G12C mutant and its effector Raf1, as well as the G12C inhibitor-induced dissociation. Furthermore, SURF also visualized the interaction between other KRas mutants including G12D and G12V with Raf1. Since G12D and G12V mutations are found in many human tumors, this demonstrates that SURF will be a useful tool for high throughput screening of small molecules that can inhibit these Ras oncoproteins.

Regulators of small GTPases include guanine nucleotide-exchange factor (GEF) and GTPase-activating protein (GAP), which activates and inhibits the interaction between a small GTPase and its effector, respectively. Indeed, SURF reports fluorescence increased and decreased upon co-expression of corresponding GEFs and GAPs of the small GTPases. SURF also detected decreased PPI by mutations that decrease GTP binding affinity and thus decreased interaction between a small GTPase and its effector.

SURF visualized interactions between transcription factors, including β-catenin and TCF4 in the wnt signaling pathway, and YAP1 and TEAD4 in the Hippo pathway. First, SURF-based reporters visualized both PPIs in the nucleus as expected. Second, co-expression of ICAT (inhibitor of β-catenin and TCF4⁴⁹⁻⁵¹) resulted in the loss of SURF fluorescence, suggesting that SURF detects inhibition of the PPI by ICAT. Addition of verteporfin, an inhibitor of YAP1 and TEAD4, led to loss of SURF fluorescence, suggesting that the SURF-based reporter for YAP1 and TEAD4 detects PPI disruption by verteporfin. Z′-factor was determined to be 0.8 (at 24 hrs), which suggests that the SURF assay was sufficiently robust for high throughput screening.

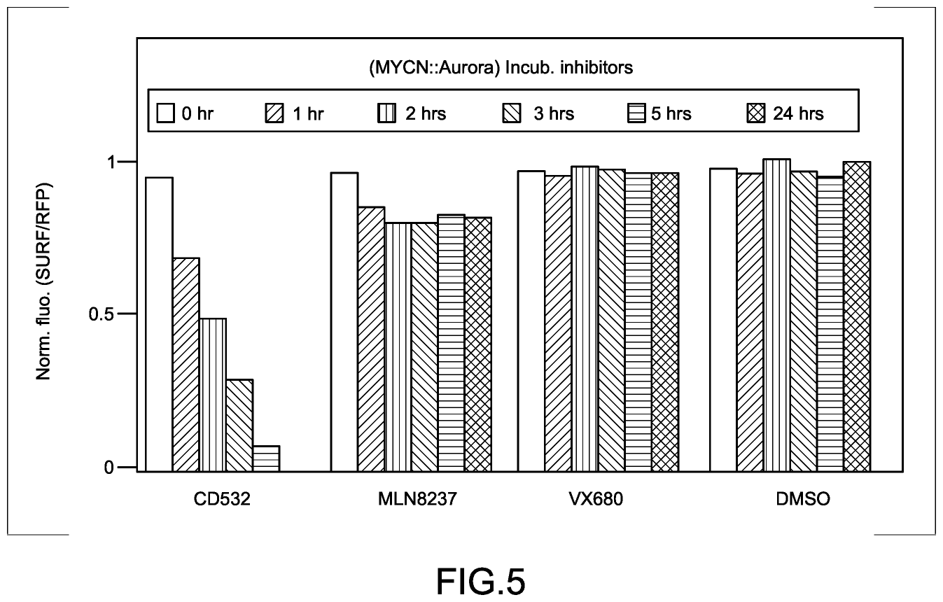

SURF visualized interactions between transcription factor and kinase, such as MYCN and aurora kinase A. SURF-based reporter of this PPI revealed punctate structures in the nucleus (FIG. 5), suggesting that the two proteins interact in the compartmentalized domains, consistent with a recent report that purified MYC proteins phase separate, forming MYC condensates. Furthermore, CD532, the small molecule inhibitor that disrupts this interaction, inhibited SURF fluorescence. But VX680, which inhibits aurora kinase activity but does not disrupt the interaction, did not inhibit the SURF fluorescence. MLN8237, which slightly disrupts the interaction, decreased SURF fluorescence by ˜5%. Thus, it was demonstrated that SURF can be used to detect PPI inhibitors. Using CD532 as the positive control and DMSO as negative control, Z′-factor was determined to be 0.8 (at both 5 hrs and 24 hrs), which suggests that the SURF assay was sufficiently robust for high throughput screening.

Other fluorogenic PPI reporters that require no exogenous cofactors are either irreversible or too dim. Other PPI reporters that are fluorogenic with large dynamic range and are genetically encoded requiring no exogenous cofactors include split iRFP, split IFP1.4, and UPPI. The latter two are reported to be reversible. All of these three reporters were tested for imaging the interaction between p53 and Mdm2. First, for the split iRFP, while its fluorescence was detected, the fluorescence did not decrease upon Nutlin-3a induced dissociation between p53 and Mdm2 within 70 minutes. As a comparison, SURF showed fluorescence loss upon addition of Nutlin-3a with OFF time ˜5 minutes. Thus, the data show that the split iRFP is not reversible. Second, for the split IFP1.4 and UPPI, no fluorescence was detected, suggesting that both reporters were too dim for imaging the interaction of p53 and Mdm2. Next, these three reporters were assessed in imaging the interaction between the KRas G12C mutant and Raf1. For split iRFP, its fluorescence was detected but did not decrease upon addition of ARS-853, even after incubation for 24 hours. For the split IFP1.4 and UPPI, no fluorescence was detected, suggesting that their brightness was too low to be useful for imaging the PPI. While there are other PCA-based fluorescent assays, none of them has been demonstrated for HTS of PPI inhibitors.

SURF-based high throughput screening of FDA-approved drug library identified agents that blocked PPI between KRas^{G12V} and Raf1. To identify clinical drugs that can inhibit the interaction between KRas^{G12V} and Raf1, a SURF-based reporter was designed for imaging this PPI. In particular, cSURF (SEQ ID NO: 8) was fused to RBD of Raf1 (Raf1RBD) and nSURF (SEQ ID NO: 6) was fused to KRas^{G12V}. SURF visualized this interaction with green fluorescence on the plasma membrane as expected. Next, a high throughput screening of 1622 FDA-approved drugs (1 μM final concentration) was performed. Six clinical drugs were identified, showing 50%-80% inhibition, left side of volcano plot (FIG. 4A). Three drugs that increase the interaction, were also identified, FIG. 4A, right side of the plot.

SURF-based high throughput screening of FDA-approved drug library identified agents that blocked PPI between YAP1 & TEAD. To identify clinical drugs that can inhibit the interaction between YAP1 and TEAD, a SURF-based reporter for imaging this PPI was developed. In particular, cSURF (SEQ ID NO: 8) was fused to YAP1 and nSURF (SEQ ID NO: 6) was fused to TEAD4. SURF visualized this interaction with fluorescence in the nucleus as expected. Furthermore, SURF detected dissociation of these two proteins by a previously reported inhibitor verteporfin. Next, a high throughput screening of 1622 FDA-approved drugs (1 μM final concentration) was carried out. Eight clinical drugs were identified that showed 50%-70% inhibition, left side of volcano plot (FIG. 4B). Also, three drugs that increase the interaction were identified, FIG. 4B right side of the plot, including, dasatinib, a SRC inhibitor. Here, ten fields-of-view (FOV) were imaged per well (i.e. per drug). The images were analyzed using ImageJ in an automated batch processing to calculate: 1) the SURF fluorescence per one FOV; 2) the co-expressed mCherry fluorescence per FOV; 3) normalized SURF fluorescence by mCherry. Then, we calculated: 1) the fold change of normalized SURF fluorescence (averaged by 10 FOV) for each drug (vs buffer); and 2) the p-value based on 10 FOV for each drug (vs buffer).

SURF-based high throughput screening of FDA-approved drug library identified agents that blocked PPI between MYCN & AURKA. To identify clinical drugs that can inhibit the interaction between MYCN and AURKA, a SURF-based reporter was developed for imaging this PPI (FIG. 5). Because this PPI occurs between the N-terminal fragment of MYCN (1-137aa) and the kinase domain of AURKA (122-403aa), cSURF (SEQ ID NO: 8) was fused to MYCN (1-137) and nSURF (SEQ ID NO: 6) was fused to AURKA (122-403). First, it was verified that SURF detected this interaction with bright green fluorescence, which is evenly distributed in the cells, in contrast to the SURF assay of the full-length MYCN and AURKA proteins. This SURF assay was ˜5-fold brighter than a SURF assay utilizing the full-length proteins. Furthermore, SURF detected dissociation of these two proteins by a previously developed PPI inhibitor CD532. CD532 not only inhibits AURKA's kinase activity by binding to the ATP site, but also inhibits AURKA's interaction with MYCN by an allosteric mechanism, inducing a conformational change in AURKA that dissociates AURKA from MYCN. VX680, a kinase inhibitor of AURKA that does not induce conformational change was also tested and did inhibit this PPI, and MLN8237, a clinical inhibitor of AURKA slightly decreased this PPI. SURF fluorescence did not change upon addition of VX680. MLN8237 slightly decreased SURF fluorescence.

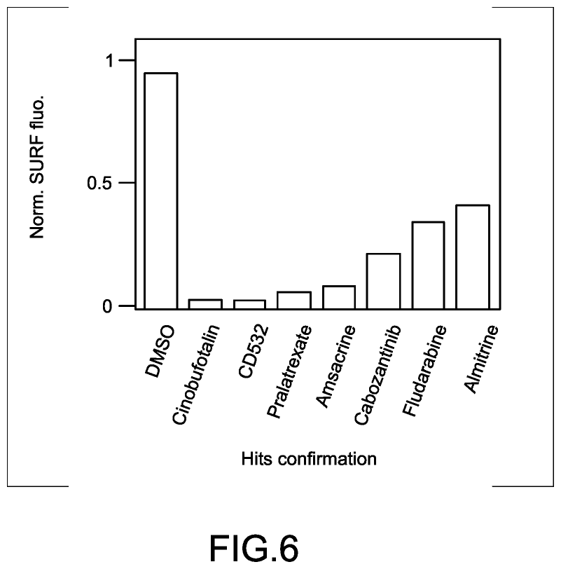

After verification of the SURF-based PPI reporter of MYCN and AURKA with inhibitory compounds and determination of Z′-factor ˜0.8 using CD532 as the positive control and DMSO as the negative control, a high throughput screening of 1622 FDA-approved drugs (1 μM final concentration) was carried out. Six clinical drugs significantly inhibited this PPI, shown in the left side of the volcano plot (FIG. 4C). One of them, cinobufotalin, inhibited the PPI at a similar degree as CD532. The hits were verified in a separate set of imaging experiments (FIG. 6). As a control, none of these drugs affected fluorescence of cSURF and nSURF that were directly linked together, suggesting that they do not perturb concentration of the endogenous bilirubin.

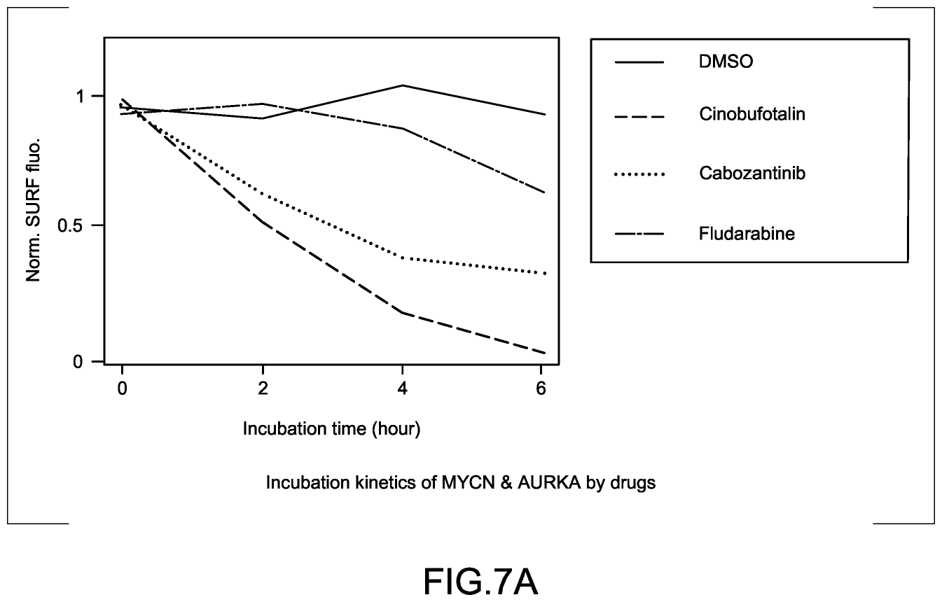

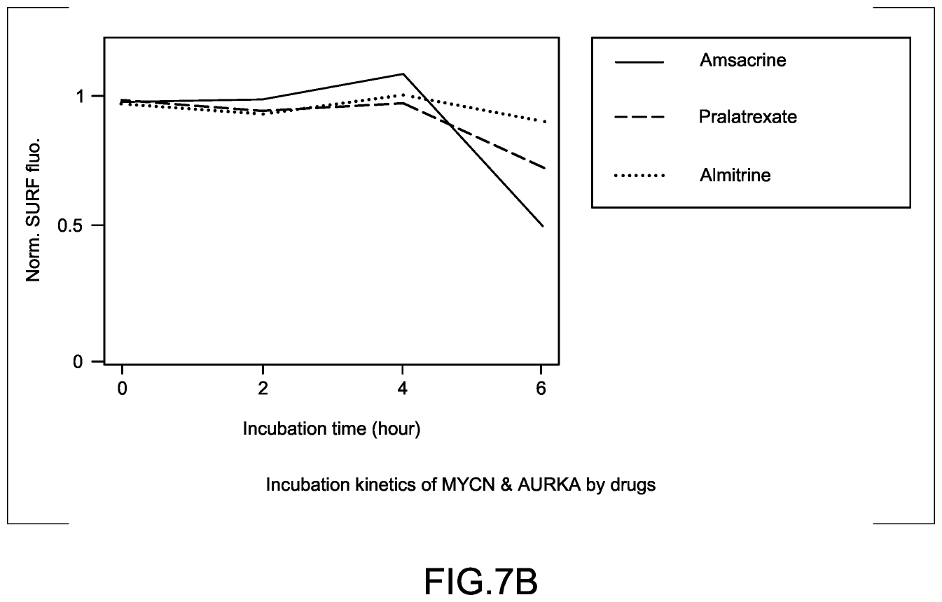

The clinical drugs inhibit PPI between MYCN and AURKA within 2-6 hours. To examine how fast the identified drugs inhibit the PPI, the top five drugs were selected for a time course assay. Imaging data showed that two of the drugs, cinobufotalin and cabozantinib, rapidly inhibited the interaction, with OFF time ˜2 and 4 hours, respectively (FIGS. 7A and 7B). Amsacrine inhibited the interaction with OFF time ˜6 hours. The other two drugs, pralatrexate and fludarabine, inhibit the interaction more slowly, >6 hours. CD532 inhibited this interaction with OFF time ˜2 hours (FIG. 5), similar to cinobufotalin.

The drugs identified degrade MYCN and inhibit proliferation of MYCN-amplified neuroblastoma cells. To examine whether the identified drugs block MYCN activity in cancer cells, MYCN protein levels were assessed by western blot. Interestingly, cinobufotalin degraded MYCN to an undetectable level, similar to CD532. Amsacrine also showed significant degradation of MYCN, albeit less than cinobufotalin. Cabozantinib showed MYCN degradation, and was the weakest among the three. The other two drugs did not affect MYCN levels.

Interestingly, while cinobufotalin and CD532 had the highest degree of MYCN degradation, they also exhibited the fastest kinetics in inhibiting the PPI. Pralatrexate and fludarabine do not degrade MYCN and also showed the slowest inhibition kinetics for the PPI. Thus, it appears that the efficiency of MYCN degradation is linked to inhibition kinetics of PPI by drugs. These data demonstrate that co-treatment with VX680 blocks the efficacy of all of the candidates, suggesting that all these clinical drugs act in-part through allosteric inhibition of Aurora Kinase A, analogous to the tool compound CD532.

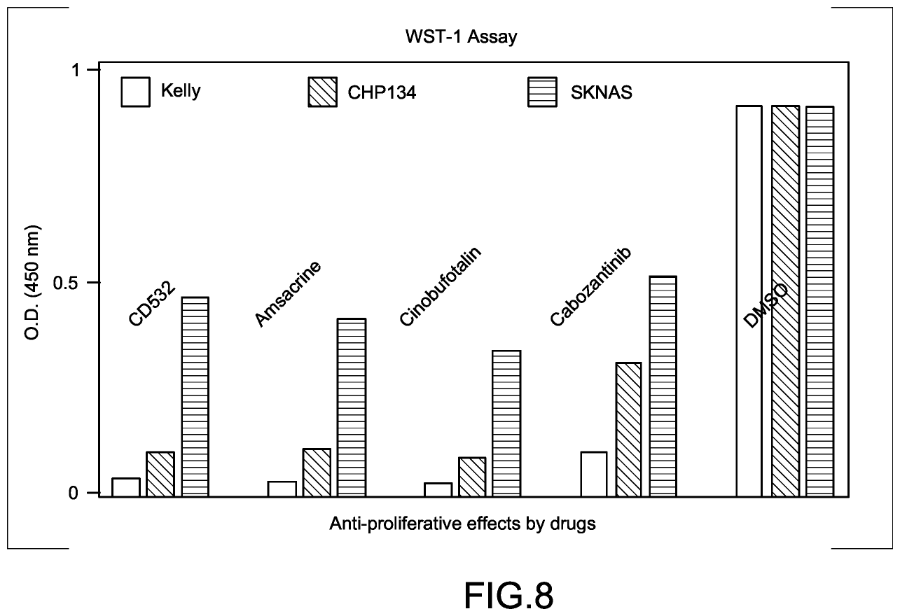

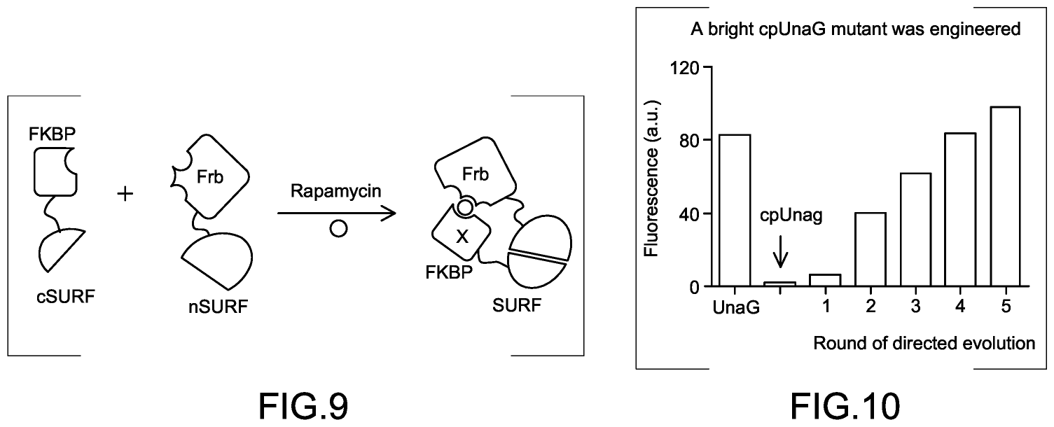

For the three drugs that degraded MYCN, their effect on cell proliferation was examined using WST-1 assay for three neuroblastoma cell lines that have different levels of MYCN amplification. Among them, Kelly cells have the highest level of MYCN amplification; CHP134 medium amplification; SKNAS no MYCN amplification (FIG. 8). All of the identified drugs and CD532 showed anti-proliferation effects for the neuroblastoma cells. Kelly cells showed the highest level of anti-proliferation effect, CHP134 medium level, and SKNAS the smallest effect. Thus, the degree of anti-proliferation appears to be proportional to the level of MYCN amplification. Furthermore, among the three drugs, cinobufotalin and amsacrine showed stronger effect on anti-proliferation than cabozantinib. This appears to be consistent with their relative effect on MYCN degradation in the Kelly cells.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

SEQUENCE LISTING
SEQ ID NO: 1
Engineered UnaG fluorescent protein.
X₁X₂X₃KFVGTWKIADSHNFGEYLX₄AIGX₅PKELSDGGDATX₆P
TLYISQKDGDKMTVKIENGPPTFLDTQVKX₇KLGEEFDEFPSDX₈
RX₉GVKSVVNLVGEKLVYVQKWDGKETTX₁₀VREIKDGKLVVTL
TMGDVVAVRSYRRATE
wherein X1 is V,G, L, A, I, T, Y, or F;
wherein X2 is L, G,A, IM, Y, or F;
wherein X3 is Q, D, N, W, H, M, S, R, or K;
wherein X4 is R, H, D, E, N, M, or Q;
wherein X5 is S, T, or A;
wherein X6 is R, T, H, D, E, N, M, or Q;
wherein X7 is L, G,A, IM, Y, or F;
wherein X8 is G, R, A, V, L, or I
wherein X9 is K or omitted; and
wherein X10 is H, D, E, N, M, or Q.
SEQ ID NO: 2
Engineered UnaG fluorescent protein.
VLQKFVGTWKIADSHNFGEYLRAIGSPKELSDGGDATRPTLYISQ
KDGDKMTVKIENGPPTFLDTQVKLKLGEEFDEFPSDGRKGVKSVV
NLVGEKLVYVQKWDGKETTHVREIKDGKLVVTLTMGDVVAVRSYR
RATE
SEQ ID NO: 3:
Engineered UnaG fluorescent protein
VLQKFVGTWKIADSHNFGEYLRAIGAPKELSDGGDATTPTLYISQ
KDGDKMTVKIENGPPTFLDTQVKFKLGEEFDEFPSDRRKGVKSVV
NLVGEKLVYVQKWDGKETTHVREIKDGKLVVTLTMGDVVAVRSYR
RATE
SEQ ID NO: 4
N-terminal Fragment of Engineered UnaG Protein
X₁X₂X₃KFVGTWKIADSHNFGEYLX₄AIGX₅PKELSDGGDATX₆PT
LYISQKDGDKMTVKIENGPPTFLDTQVKX₇KLGEEFDEFPSDX₈R,
wherein X1 is V,G, L, A, I, T, Y, or F;
wherein X2 is L, G,A, IM, Y, or F;
wherein X3 is Q, D, N, W, H, M, S, R, or K;
wherein X4 is R, H, D, E, N, M, or Q;
wherein X5 is S, T, or A;
wherein X6 is R, T, H, D, E, N, M, or Q;
wherein X7 is L, G,A, IM, Y, or F; and
wherein X8 is G, R, A, V, L, or I.
SEQ ID NO: 5:
N-terminal Fragment of Engineered UnaG Protein
VLQKFVGTWKIADSHNFGEYLRAIGSPKELSDGGDATRPTLYIS
QKDGDKMTVKIENGPPTFLDTQVKLKLGEEFDEFPSDGR
SEQ ID NO: 6:
N-terminal Fragment of Engineered UnaG Protein
VLQKFVGTWKIADSHNFGEYLRAIGAPKELSDGGDATTPTLYIS
QKDGDKMTVKIENGPPTFLDTQVKFKLGEEFDEFPSDRR
SEQ ID NO: 7:
C-terminal Fragment of Engineered UnaG Protein
GVKSVVNLVGEKLVYVQKWDGKETTX₁VREIKDGKLVVTLTMGD
VVAVRSYRRATE,
wherein X1 is H, D, E, N, M, or Q.
SEQ ID NO: 8:
C-terminal Fragment of Engineered UnaG Protein
GVKSVVNLVGEKLVYVQKWDGKETTHVREIKDGKLVVTLTMGDVV
AVRSYRRATE
SEQ ID NO: 9
Linker sequence
GGSA
SEQ ID NO: 10
Nucleic Acid sequence coding for N-terminal
Fragment of Engineered UnaG Protein
gtgctccagaaattcgtaggaacttggaagatagcagattcacat
aatttoggcgaatatctcagagccataggagcaccaaaggaatta
tcagatggcggtgatgccacaactcccactctgtatatcagccag
aaagatggcgacaaaatgacggtgaaaatagagaacggcccaccg
accttcctggacactcaggtgaagtttaaactgggtgaggagttt
gacgagtttccttctgacgggcgt.
SEQ ID NO: 11:
Nucleic Acid sequence coding for C-terminal
Fragment of Engineered UnaG Protein
ggtgttaaaagcgtcgttaacctggtgggagagaaattagtatac
gtccaaaagtgggacggcaaggagactacgcacgttagagagatt
aaagacggcaagctggtggtaacactaacaatgggcgacgtcgtt
gcagtgcgctcatatcgcagggcgacggag

What is claimed is:

1. An engineered UnaG protein comprising a protein having at least 95% sequence identity to SEQ ID NO: 1.

2. The engineered UnaG protein of claim 1, comprising
a protein having at least 95% sequence identity to SEQ ID NO: 2 and comprising valine at amino acid position 1, leucine at amino acid position 2, glutamine at amino acid position 3, arginine at amino acid position 22, serine at amino acid position 26, arginine at amino acid position 38, leucine at amino acid position 69, glycine at amino acid position 82, and histidine at amino acid position 110.

3. The engineered UnaG protein of claim 1, comprising
a protein having at least 95% sequence identity to SEQ ID NO: 3 and comprising valine at amino acid position 1, leucine at amino acid position 2, glutamine at amino acid position 3, arginine at amino acid position 22, and histidine at amino acid position 110.

4. A protein comprising
an amino acid sequence having at least 95% sequence identity to SEQ ID NO: 4.

5. A protein comprising
an amino acid sequence having at least 95% sequence identity to SEQ ID NO: 5.

6. A protein comprising
an amino acid sequence having at least 95% sequence identity to SEQ ID NO: 6.

7. A protein comprising
an amino acid sequence having at least 95% sequence identity to SEQ ID NO: 7.

8. A protein comprising
an amino acid sequence having at least 95% sequence identity to SEQ ID NO: 8.

9. A BiFC assay comprising
a first BiFC construct comprising a first fluorescent protein fragment joined to a first interacting partner; and
a second BiFC construct comprising a second fluorescent protein fragment, joined to a second interacting partner;
wherein the first and second fluorescent protein fragments comprise complementary fragments of an engineered UnaG protein, wherein the engineered UnaG protein comprises a sequence having at least 95%, sequence identity to SEQ ID NO: 1, SEQ ID NO: 2, or SEQ ID NO: 3;
wherein, interaction of the first and second interacting partners brings the first and second fluorescent protein fragments into sufficient proximity to generate a fluorescent signal in response to illumination with a suitable wavelength of light.

10. The BiFC assay of claim 9, wherein
the engineered UnaG protein comprises SEQ ID NO: 3.

11. The BiFC assay of claim 9, wherein
the first fluorescent protein fragment comprises amino acids 1-70 of the engineered UnaG protein; and
the second fluorescent protein comprises amino acids 90-139 of the engineered UnaG protein.

12. The BiFC assay of claim 11, wherein
the first fluorescent protein fragment comprises amino acids 1-83 of the engineered UnaG protein; and
the second fluorescent protein comprises amino acids 85-139 of the engineered UnaG protein.

13. The BiFC assay of claim 11, wherein
the first fluorescent protein fragment comprises an amino acid sequence having at least 95% sequence identity to SEQ ID NO: 4, SEQ ID NO: 5, or SEQ ID NO: 6; and
the second fluorescent protein comprises an amino acid sequence having at least 95% sequence identity to SEQ ID NO: 7 or SEQ ID NO: 8.

14. The BiFC assay of claim 9, wherein
one or both of the first and second interacting partners are proteins.

15. The BiFC assay of claim 9, wherein
the first and second interacting partners are proteins;
the first BiFC construct comprises a fusion protein comprising the first interacting partner fused to the first fluorescent protein fragment; and
the second BiFC construct comprises a fusion protein comprising the second interacting partner fused to the second fluorescent protein fragment.

16. The BiFC assay of claim 14, wherein
the first and second BiFC constructs are expressed in a cell.

17. A cell,
wherein the cell is engineered to express a first BiFC construct and a second BiFC construct, wherein
the first BiFC construct comprises a first PPI partner of a selected PPI fused to a first fluorescent protein fragment; and
the second BiFC construct comprises a second PPI partner of the selected PPI fused to a second fluorescent protein fragment;
wherein the first and second fluorescent fragments comprise complementary fragments of an engineered UnaG protein, the engineered UnaG protein comprising a protein having at least 95% sequence identity to SEQ ID NO: 1, SEQ ID NO: 2, or SEQ ID NO: 3;
wherein, interaction of the first and second PPI partners brings the first and second fluorescent protein fragments into sufficient proximity to generate a fluorescent signal in response to illumination with a suitable wavelength of light.

18. The cell of claim 17, wherein
the engineered UnaG protein comprises SEQ ID NO: 3.

19. The cell of claim 17, wherein
the first fluorescent protein fragment comprises amino acids 1-70 of the engineered UnaG protein; and
the second fluorescent protein fragment comprises amino acids 90-139 of the engineered UnaG protein.

20. The cell of claim 19, wherein
the first fluorescent protein fragment comprises amino acids 1-83 of the engineered UnaG protein; and
the second fluorescent protein fragment comprises amino acids 85-139 of engineered UnaG protein.

21. The cell of claim 19, wherein
the first fluorescent protein fragment comprises SEQ ID NO: 4 and the second fluorescent protein fragment comprises SEQ ID NO: 7.

22. The cell of claim 19, wherein
the first fluorescent protein fragment comprises SEQ ID NO: 5 and the second fluorescent protein fragment comprises SEQ ID NO: 8.

23. The cell of claim 19, wherein
the first fluorescent protein fragment comprises SEQ ID NO: 6 and the second fluorescent protein fragment comprises SEQ ID NO: 8.

24. The cell of claim 17, wherein
the cell comprises any of a bacterial cell, a yeast cell, an insect cell, an animal cell, a mammalian cell, or a plant cell, a HEK293 cell, a HeLa cell, a Jurkat cell, a PC3 cell, a cell of a cancer cell line, a CHO cell, an Sf9 cell, an E. coli cell, an Ns0 cell, an Sp2/0 cell; a cultured cell, a cell present in a tissue or organ of a living organism or an explant thereof, and a cell in an organoid.

25. The cell of claim 17, wherein
the cell comprises a detection system for detecting a PPI between KRas^{G12V} and Raf1,
wherein the first PPI partner comprises a protein comprising KRas^{G12V} or a subsequence thereof which interacts with Raf1; and
wherein the second PPI partner comprises Raf1 or a subsequence thereof which interacts with KRas^{G12V}.

26. The cell of claim 17, wherein
the cell comprises a detection system for detecting a PPI between YAP1 and TEAD;
wherein the first PPI partner comprises a protein comprising YAP1 or a subsequence thereof which interacts with TEAD; and
wherein the second PPI partner comprises TEAD or a subsequence thereof which interacts with YAP1.

27. The cell of claim 17, wherein
the cell comprises a detection system for detecting a PPI between MYCN and AURKA, wherein
wherein the first PPI partner comprises a protein comprising MYCN or a subsequence thereof which interacts with AURKA; and
wherein the second PPI partner comprises AURKA or a subsequence thereof which interacts with MYCN.
